(19) United States
(12) Patent Application Publication
(10) Pub. No.: US 2025/0368644 A1
(43) Pub. Date: Dec. 4, 2025

(54) DURLOBACTAM CRYSTALLINE FORMS

(71) Applicants: Thomas Francois DURAND-REVILLE, Belmont, MA (US); Frank WU, Nanjing (CN); Xiaoming LIAO, Cheshire (GB); Xiaona WANG, Cheshire (GB); Sixing ZHANG, Cheshire (GB); Entasis Therapeutics Limited, Cheshire (GB)

(72) Inventors: Thomas Francois Durand-Reville, Belmont, MA (US); Frank Wu, Nanjing (CN); Xiaoming Liao, Cheshire (GB); Xiaona Wang, Cheshire (GB); Sixing Zhang, Cheshire (GB); Matthew Ronsheim, Sudbury, MA (US); Kishore Ramachandran, Westford, MA (US)

(21) Appl. No.: 18/860,970

(22) PCT Filed: Apr. 28, 2023
(86) PCT No.: PCT/US2023/020439

§ 371 Date: Oct. 28, 2024

(30) Foreign Application Priority Data
Apr. 29, 2022 (WO) PCT/CN2022/090815

(51) Int. Cl. C07D 471/08

(52) U.S. Cl. CPC C07D 471/08

(57) ABSTRACT

Provided herein are salt forms of Durlobactam (DUR) having the Formula I: In particular, crystalline forms of a DUR-TBA, a DUR-TEA, and a DUR-Ca are provided. The methods of preparing these salts, and characterization of their various polymorphic forms are also provided. Additionally, the present invention comprises methods for synthesizing the DUR-Na from the various crystalline DUR salts disclosed.

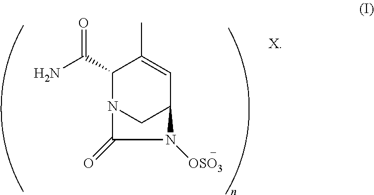

### RELATED APPLICATIONS

This application claims priority to International Application No. PCT/CN2022/090815, filed Apr. 29, 2022, the entire contents of which are incorporated herein by reference.

### BACKGROUND

Durlobactam (DUR; previously designated ETX2514) is a novel, broad-spectrum and potent inhibitor of Class A, C, and D B-lactamases. Sulbactam (SUL) is a β-lactam antibiotic with activity against Acinetobacter baumannii; however, β-lactamase-mediated resistance to sulbactam is now widespread rendering it generally ineffective. In preclinical studies, durlobactam was found to inhibit the β-lactamases commonly found in A. baumannii thus restoring sulbactam's activity. Currently, a SUL-DUR combination product (also designated Sulbactam-Durlobactam) is being developed for the treatment of serious infections caused by Acinetobacter, including multidrug-resistant (MDR) strains.

The sodium salt of DUR is the active pharmaceutical ingredient used for intravenous injection and is described in Example 10 of WO 2013/150296. The process for making the sodium salt of DUR includes the step of first forming a phosphonium salt which is then exchanged to sodium via ion-exchange resin. However, the phosphonium salt cannot be crystallized and its purity is less than 95%. In addition, it is not amendable to large scale batches (e.g., multi-kilograms), which is necessary for expansive production.

Accordingly, chemical precursors and methods which allow for the large-scale production of DUR, particularly its sodium salt, are needed.

### SUMMARY

Provided herein are crystalline forms of durlobactam that can be used for the large-scale preparation of the sodium salt of durlobactam. Such crystalline forms include those having the Formula I

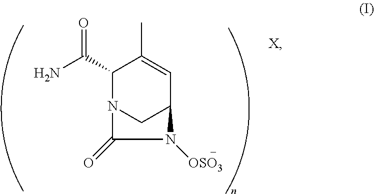

where X and n are as defined herein.

In one aspect, the crystalline forms described herein include a Durlobactam Tetrabutylammonium salt (DUR-TBA), Durlobactam Triethylammonium salt (DUR-TEA), Durlobactam Calcium salt (DUR-Ca), each of which, unlike the prior described phosphonium salt from Example 10 of WO 2013/150296, were found to be suitable for multi-kilogram preparation of Durlobactam Sodium salt (DUR-Na).

Also provided herein are polymorphic forms of the disclosed DUR-TBA, DUR-TEA, DUR-Ca.

Further provided are methods for making the disclosed DUR-TBA, DUR-TEA, DUR-Ca, as well as their polymorphic forms.

Further provided are methods of making DUR-Na from the disclosed DUR-TBA, DUR-TEA, DUR-Ca, as well as their polymorphic forms.

### BRIEF DESCRIPTION OF THE DRAWINGS

FIG. 1. XRPD of DUR-TBA Form A

FIG. 2. TGA and DSC of DUR-TBA Form A

FIG. 3. XRPD of DUR-TEA Form A

FIG. 4. TGA and DSC of DUR-TEA Form A

FIG. 5. XRPD of DUR-Ca Form B

FIG. 6. TGA and DSC of DUR-Ca Form B

FIG. 7. XRPD of DUR-Ca Form A

FIG. 8. TGA of DUR-Ca Form A

FIG. 9. DSC of DUR-Ca Form A

FIG. 10. XRPD of DUR-Ca Form C

FIG. 11. TGA of DUR-Ca Form C

FIG. 12. DSC of DUR-Ca Form C

FIG. 13. XRPD of DUR-Ca Form F

FIG. 14. TGA and DSC of DUR-Ca Form F

FIG. 15. Summary of DUR-Ca Crystalline Forms

### DETAILED DESCRIPTION

Provided are salt forms of DUR having the Formula I

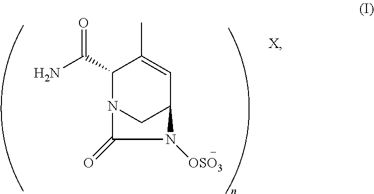

wherein
n is 1 or 2; and
X is a positively charged amine or a Ca, Mg, Zn, K, Na, Li, Cs, Ba, Rb, Sr, Fe, Co, Ni, Cu, Zn, Ag, or Au cation.

As used herein, “crystalline” refers to a solid form of DUR where the atoms form a three-dimensional arrangement within a single repeating unit called a unit cell. The crystalline nature of DUR can be confirmed, for example, by examination of the X-ray powder diffraction pattern.

A “single crystalline form” means that DUR is present as a single crystal or a plurality of crystals in which each crystal has the same crystal form. Percent by weight of a particular crystal form is determined by the weight of the particular crystal form divided by the sum weight of the particular crystal, plus the weight of the other crystal forms present plus the weight of amorphous form present multiplied by 100%. “Pure single crystalline form” means that DUR is present as a single crystal or a plurality of crystals in which each crystal has the same crystal form with no other detectable amounts of other crystal forms present.

Chemical purity refers to extent by which the disclosed form is free from materials having different chemical structures. Chemical purity of DUR in the disclosed crystal forms means the weight of DUR divided by the sum of the weight of DUR plus materials/impurities having different chemical structures multiplied by 100%, i.e., percent by weight.

The term “amorphous” refers to DUR present in a non-crystalline state or form. Amorphous solids are disordered arrangements of molecules and therefore possess no distinguishable crystal lattice or unit cell and consequently have no definable long-range ordering. Solid state ordering of solids may be determined by standard techniques known in the art, e.g., by X-ray powder diffraction (XRPD) or differential scanning calorimetry (DSC).

The 2-theta (2Θ) values of the X-ray powder diffraction patterns for the crystalline forms described herein may vary slightly from one instrument to another and also depending on variations in sample preparation and batch to batch variation due to factors such as temperature variation, sample displacement, and the presence or absence of an internal standard. Therefore, unless otherwise defined, the XRPD patterns/assignments recited herein are not to be construed as absolute and can vary±0.2 degrees. It is well known in the art that this variability will account for the above factors without hindering the unequivocal identification of a crystal form. Unless otherwise specified, the 2-theta values provided herein were obtained using Cu Ka1 radiation.

Temperature values, e.g., for DSC peaks herein may vary slightly from one instrument to another and also depending on variations in sample preparation, batch to batch variation, and environmental factors. Therefore, unless otherwise defined, temperature values recited herein are not to be construed as absolute and can vary ±5 degrees or ±2 degrees.

“Substantially the same XRPD pattern” or “an X-ray powder diffraction pattern substantially similar to” a defined figure means that for comparison purposes, at least 90% of the peaks shown are present. It is to be further understood that for comparison purposes some variability in peak intensities from those shown are allowed, such as ±0.2 degrees.

In a first embodiment, X in the salt of Formula I is a positively charged amine or a Ca cation. Alternatively, as part of a first embodiment, X in the salt of Formula I is a positively charged amine. In another alternative, as part of a first embodiment, X in the salt of Formula I is a tertiary amine or a quaternary amine. In another alternative, as part of a first embodiment, X in the salt of Formula I is trimethylammonium, tricthylammonium, tributylammonium, triisopropylammonium, or N,N-diisopropylethylammonium. In another alternative, as part of a first embodiment, X in the salt of Formula I is triethylammonium.

In a second embodiment, the salt of Formula I is of the structural formula:

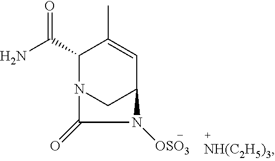

herein referred to as Durlobactam Tricthylammonium salt (DUR-TEA).

In a third embodiment, the salt of Formula I or (DUR-TEA) is crystalline.

In a fourth embodiment, DUR-TEA is of crystalline Form A. Alternatively, as part of a fourth embodiment, DUR-TEA is of crystalline Form A characterized by at least three x-ray powder diffraction peaks at 2Θ angles selected from 9.5°, 10.7°, 12.7°, 13.5°, 17.3°, 22.6°, and 24.4°. In another alternative, as part of a fourth embodiment, DUR-TEA is of crystalline Form A characterized by at least four x-ray powder diffraction peaks at 2Θ angles selected from 9.5°, 10.7°, 12.7°, 13.5°, 17.3°, 22.6°, and 24.4°. In another alternative, as part of a fourth embodiment, DUR-TEA is of crystalline Form A characterized by at least five x-ray powder diffraction peaks at 2Θ angles selected from 9.5°, 10.7°, 12.7°, 13.5°, 17.3°, 22.6°, and 24.4°. In another alternative, as part of a fourth embodiment, DUR-TEA is of crystalline Form A characterized by at least six x-ray powder diffraction peaks at 2Θ angles selected from 9.5°, 10.7°, 12.7°, 13.5°, 17.3°, 22.6°, and 24.4°. In another alternative, as part of a fourth embodiment, DUR-TEA is of crystalline Form A characterized by x-ray powder diffraction peaks at 2Θ angles 9.5°, 10.7°, 12.7°, 13.5°, 17.3°, 22.6°, and 24.4°. In another alternative, as part of a fourth embodiment, DUR-TEA is of crystalline Form A characterized by at least three, at least four, at least five, at least six, or at least seven x-ray powder diffraction peaks at 2Θ angles recited in Table 16.

In a fifth embodiment, DUR-TEA crystalline Form A is at least 70% a single crystalline form by weight, at least 80% a single crystalline form by weight, at least 90% a single crystalline form by weight, at least 95% a single crystalline form by weight, or at least 99% a single crystalline form by weight optionally characterized by the XRPD peaks recited above in the fourth embodiment. Alternatively, as part of a fifth embodiment, DUR-TEA crystalline Form A is present in pure crystalline form optionally characterized by the XRPD peaks recited above in the fourth embodiment.

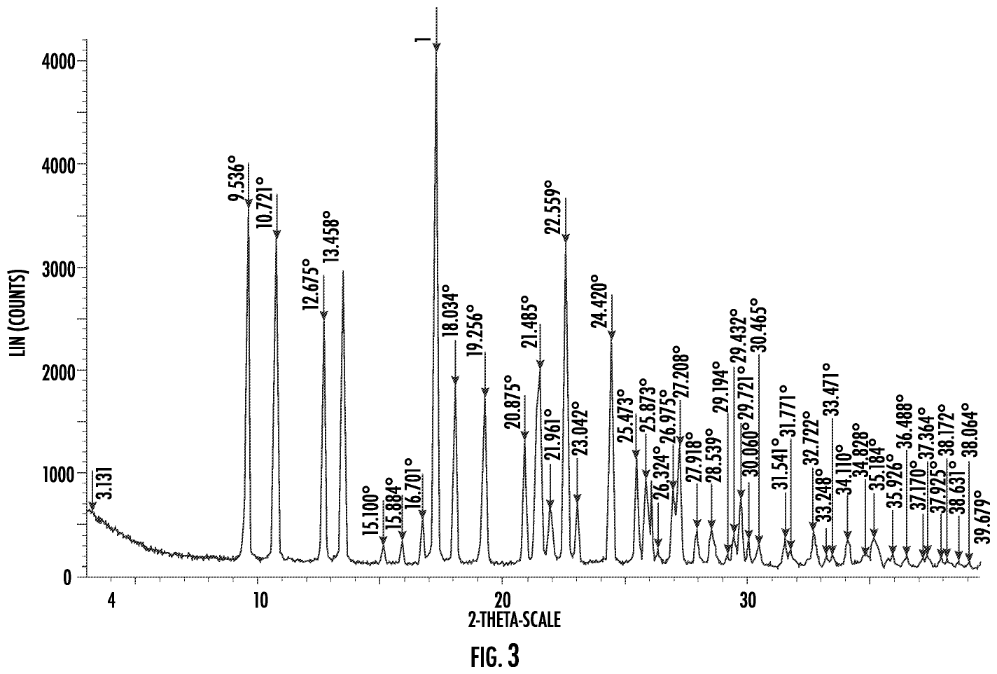

In a sixth embodiment, DUR-TEA crystalline Form A is characterized by an X-ray powder diffraction pattern substantially similar to FIG. 3.

In a seventh embodiment, X in the salt of Formula I is tetrabutylammonium, tetraethylammonium, tetramethylammonium, or tetrapropylammonium. Alternatively, as part of a seventh embodiment, X in the salt of Formula I is tetrabutylammonium.

In an eighth embodiment, the salt of Formula I is of the structural formula:

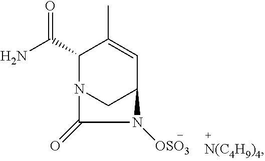

herein referred to as Durlobactam Tetrabutylammonium salt (DUR-TBA).

In a ninth embodiment, the salt of Formula I or DUR-TBA is crystalline.

In a tenth embodiment, DUR-TBA is of crystalline Form A. Alternatively, as part of a tenth embodiment, DUR-TBA is of crystalline Form A, characterized by at least three x-ray powder diffraction peaks at 2Θ angles selected from 7.3°. 8.5°. 8.7°,10.3°, 12.7°, 19.5° and 21.4°. In another alternative, as part of a tenth embodiment, DUR-TBA is of crystalline Form A, characterized by at least four x-ray powder diffraction peaks at 2Θ angles selected from 7.3°, 8.5°, 8.7°0.10.3°, 12.7°, 19.5° and 21.4°. In another alternative, as part of a tenth embodiment, DUR-TBA is of crystalline Form A, characterized by at least five x-ray powder diffraction peaks at 2Θ angles selected from 7.3°, 8.5°. 8.7°, 10.3°, 12.7°, 19.5° and 21.4°. In another alternative, as part of a tenth embodiment, DUR-TBA is of crystalline Form A, characterized by at least six x-ray powder diffraction peaks at 2Θ angles selected from 7.3°, 8.5°. 8.7°,10.3°, 12.7°, 19.5° and 21.4°. In a tenth embodiment as part of a tenth embodiment, DUR-TBA is of crystalline Form A, characterized by x-ray powder diffraction peaks at 2Θ angles selected from 7.3°. 8.5°, 8.7°, 10.3°, 12.7°, 19.5° and 21.4°. In another alternative, as part of a tenth embodiment, DUR-TBA is of crystalline Form A, characterized by at least three, at least four, at least five, at least six, or at least seven x-ray powder diffraction peaks at 2Θ angles recited in Table 15.

In an eleventh embodiment, DUR-TBA crystalline Form A is at least 70% a single crystalline form by weight, at least 80% a single crystalline form by weight, at least 90% a single crystalline form by weight, at least 95% a single crystalline form by weight, or at least 99% a single crystalline form by weight optionally characterized by the XRPD peaks recited above in the tenth embodiment. Alternatively, as part of an eleventh embodiment, DUR-TBA crystalline Form A is present in pure crystalline form optionally characterized by the XRPD peaks recited above in the tenth embodiment.

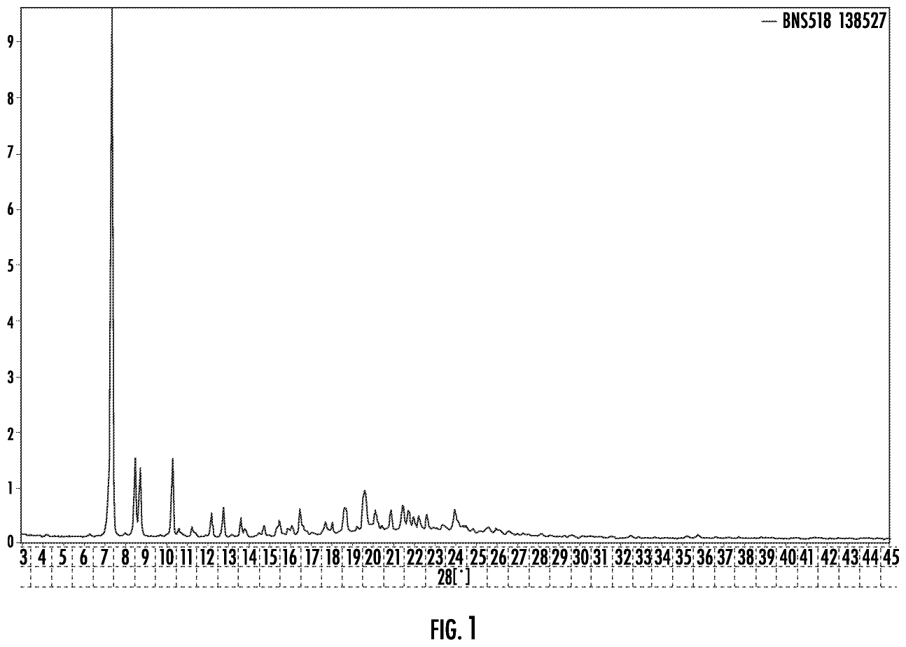

In a twelfth embodiment, DUR-TBA crystalline Form A is characterized by an x-ray powder diffraction pattern substantially similar to FIG. 1.

In a thirteenth embodiment, the salt of Formula I is of the structural formula:

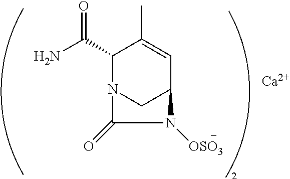

herein referred to as Durlobactam Calcium salt (DUR-Ca).

In a fourteenth embodiment, the salt of Formula I or (DUR-Ca) is crystalline.

In a fifteenth embodiment, DUR-Ca is of crystalline Form B. Alternatively, as part of a fifteenth embodiment, DUR-Ca is of crystalline Form B, characterized by at least three x-ray powder diffraction peaks at 2Θ angles selected from 9.6°, 12.5°, 12.7°, 14.1°, 16.5°, 16.6, 22.5°, and 24.6°. In another alternative, as part of a fifteenth embodiment, DUR-Ca is of crystalline Form A, characterized by at least four x-ray powder diffraction peaks at 2Θ angles selected from 9.6°, 12.5°, 12.7°, 14.1°, 16.5°, 16.6, 22.5°, and 24.6°. In another alternative, as part of a fifteenth embodiment, DUR-Ca is of crystalline Form A, characterized by at least five x-ray powder diffraction peaks at 2Θ angles selected from 9.6°, 12.5°. 12.7°, 14.1°, 16.5°, 16.6, 22.5°, and 24.6°. In another alternative, as part of a fifteenth embodiment, DUR-Ca is of crystalline Form A, characterized by at least six x-ray powder diffraction peaks at 2Θ angles selected from 9.6°, 12.5°, 12.7°, 14.1°, 16.5°, 16.6, 22.5°, and 24.6°. In another alternative, as part of a fifteenth embodiment, DUR-Ca is of crystalline Form A, characterized by x-ray powder diffraction peaks at 2Θ angles selected from 9.6°, 12.5°. 12.7°, 14.1°, 16.5°, 16.6, 22.5°, and 24.6°. In another alternative, as part of a fifteenth embodiment, DUR-Ca is of crystalline Form B, characterized by at least three, at least four, at least five, at least six, or at least seven x-ray powder diffraction peaks at 2Θ angles recited in Table 17.

In a sixteenth embodiment, DUR-Ca crystalline Form B is at least 70% a single crystalline form by weight, at least 80% a single crystalline form by weight, at least 90% a single crystalline form by weight, at least 95% a single crystalline form by weight, or at least 99% a single crystalline form by weight optionally characterized by the XRPD peaks recited above in the fifteenth embodiment. Alternatively, as part of a sixteenth embodiment, DUR-Ca crystalline Form A is present in pure crystalline form optionally characterized by the XRPD peaks recited above in the sixteenth embodiment.

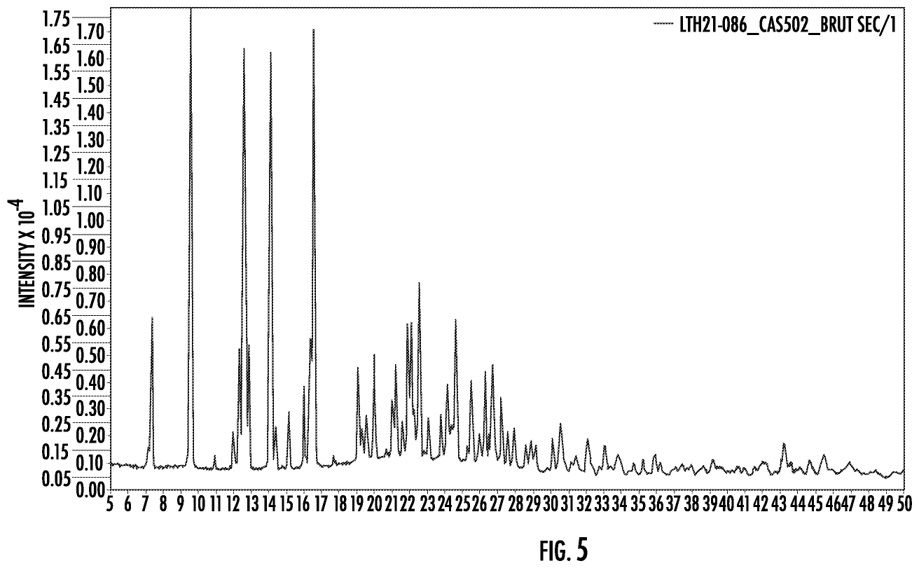

In a seventeenth embodiment, DUR-Ca crystalline Form B is characterized by an X-ray powder diffraction pattern substantially similar to FIG. 5.

In an eighteenth embodiment, DUR-Ca is of crystalline Form A. Alternatively, as part of an eighteenth embodiment, DUR-Ca is of crystalline Form A, characterized by at least three x-ray powder diffraction peaks at 2Θ angles selected from 7.8°, 9.0°, 11.9°, 13.4°, 16.2°, 19.5°, 20.5°, and 25.0°. In another alternative, as part of an eighteenth embodiment, DUR-Ca is of crystalline Form A, characterized by at least four x-ray powder diffraction peaks at 2Θ angles selected from 7.8°, 9.0°, 11.9°, 13.4°, 16.2°, 19.5°, 20.5°, and 25.0°. In another alternative, as part of an eighteenth embodiment, DUR-Ca is of crystalline Form A, characterized by at least five x-ray powder diffraction peaks at 2Θ angles selected from 7.8°, 9.0°, 11.9°, 13.4°, 16.2°, 19.5°, 20.5°, and 25.0°. In another alternative, as part of an eighteenth embodiment, DUR-Ca is of crystalline Form A, characterized by at least six x-ray powder diffraction peaks at 2Θ angles selected from 7.8°, 9.0°, 11.9°, 13.4°, 16.2°, 19.5°, 20.5°, and 25.0°. In another alternative, as part of an eighteenth embodiment, DUR-Ca is of crystalline Form A, characterized by x-ray powder diffraction peaks at 2Θ angles selected from 7.8°, 9.0°, 11.9°, 13.4°, 16.2°, 19.5°, 20.5°, and 25.0°. In another alternative, as part of an eighteenth embodiment, DUR-Ca is of crystalline Form A, characterized by at least three, at least four, at least five, at least six, or at least seven x-ray powder diffraction peaks at 2Θ angles recited in Table 18.

In a nineteenth embodiment, DUR-Ca crystalline Form A is at least 70% a single crystalline form by weight, at least 80% a single crystalline form by weight, at least 90% a single crystalline form by weight, at least 95% a single crystalline form by weight, or at least 99% a single crystalline form by weight optionally characterized by the XRPD peaks recited above in the fifteenth embodiment. Alternatively, as part of a nineteenth embodiment, DUR-Ca crystalline Form A is present in pure crystalline form optionally characterized by the XRPD peaks recited above in the sixteenth embodiment.

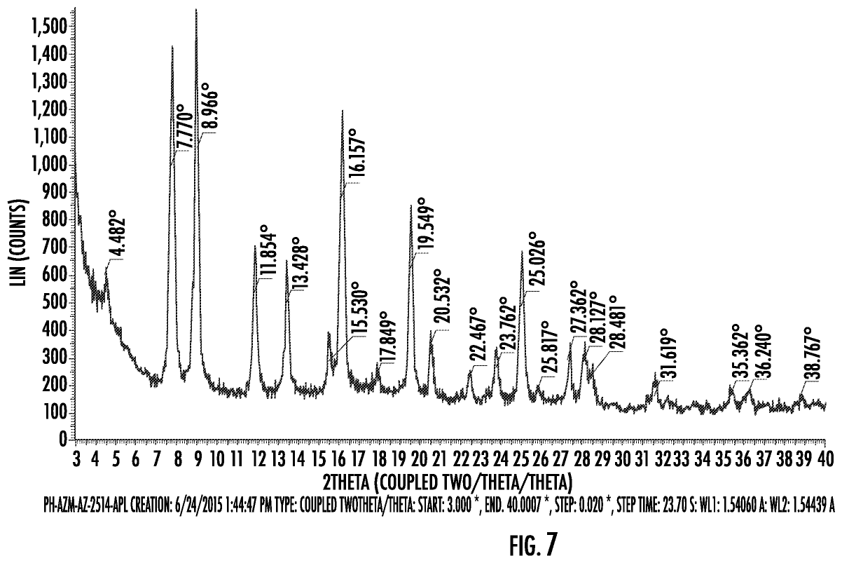

In a twentieth embodiment, DUR-Ca crystalline Form A is characterized by an X-ray powder diffraction pattern substantially similar to FIG. 7.

In a twenty-first embodiment, the salt of DUR-Ca is of crystalline Form C. Alternatively, as part of a twenty-first embodiment, DUR Ca is of crystalline Form C, characterized by at least three x-ray powder diffraction peaks at 2Θ angles selected from 7.0°, 9.5°, 12.1°, 16.1°, 16.9°, 19.7°, 20.3°, and 26.9°. In another alternative, as part of a twenty-first embodiment, DUR-Ca is of crystalline Form C, characterized by at least four x-ray powder diffraction peaks at 2Θ angles selected from 7.0°, 12.2°, 16.1°, 16.9°, 19.7°, 20.3°, and 26.9°. In another alternative, as part of a twenty-first embodiment, DUR-Ca is of crystalline Form C, characterized by at least five x-ray powder diffraction peaks at 2Θ angles selected from 7.0°, 12.2°, 16.1°, 16.9°, 19.7°, 20.3°, and 26.9°. In another alternative, as part of a twenty-first embodiment, DUR-Ca is of crystalline Form C, characterized by at least six x-ray powder diffraction peaks at 2Θ angles selected from 7.0°, 12.2°, 16.1°. 16.9°, 19.7°, 20.3°, and 26.9°. In another alternative, as part of a twenty-first embodiment, DUR-Ca is of crystalline Form C, characterized by x-ray powder diffraction peaks at 2Θ angles selected from 7.0°, 12.2°, 16.1°, 16.9°, 19.7°, 20.3°, and 26.9°. In another alternative, as part of a twenty-first embodiment, DUR-Ca is of crystalline Form C, characterized by at least three, at least four, at least five, at least six, or at least seven x-ray powder diffraction peaks at 2Θ angles recited in Table 20.

In a twenty-second embodiment, DUR-Ca crystalline Form C is at least 70% a single crystalline form by weight, at least 80% a single crystalline form by weight, at least 90% a single crystalline form by weight, at least 95% a single crystalline form by weight, or at least 99% a single crystalline form by weight optionally characterized by the XRPD peaks recited above in the eighteenth embodiment. Alternatively, as part of a twenty-second embodiment, DUR-Ca crystalline Form C is present in pure crystalline form optionally characterized by the XRPD peaks recited above in the eighteenth embodiment.

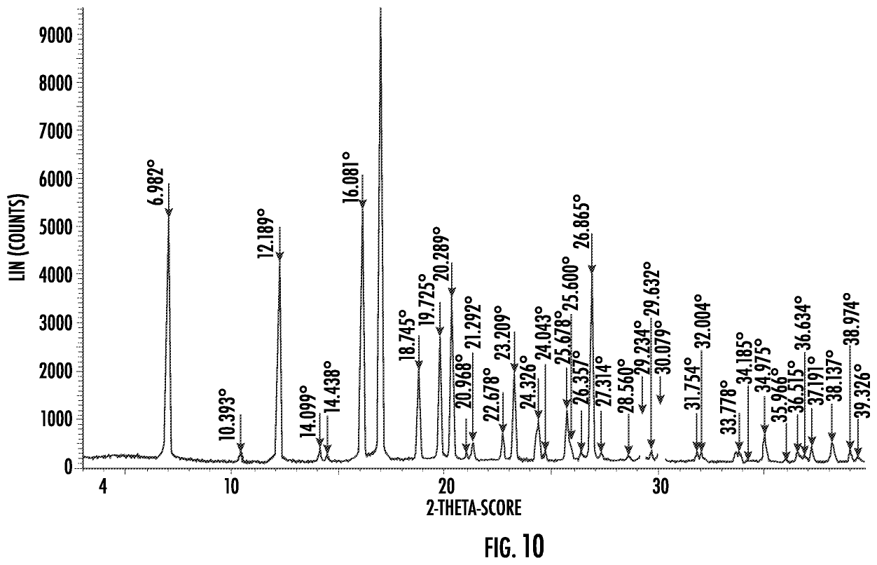

In a twenty-third embodiment, DUR-Ca crystalline Form C is characterized by an X-ray powder diffraction pattern substantially similar to FIG. 10.

In a twenty-fourth embodiment, the salt of DUR-Ca is of crystalline Form F. Alternatively, as part of a twenty-fourth embodiment, DUR Ca is of crystalline Form F, characterized by at least three x-ray powder diffraction peaks at 2Θ angles selected from 9.5°, 11.3°, 12.0°, 14.0°, 17.0°, 19.0°, and 19.5°. In another alternative, as part of a twenty-fourth embodiment, DUR-Ca is of crystalline Form F, characterized by at least four x-ray powder diffraction peaks at 2Θ angles selected from 9.5°, 11.3°. 12.0°, 14.0°, 17.0°, 19.0°, 22.3°, and 24.2°. In another alternative, as part of a twenty-fourth embodiment, DUR-Ca is of crystalline Form F, characterized by at least five x-ray powder diffraction peaks at 2Θ angles selected from 9.5°. 11.3°, 12.0°, 14.0°, 17.0°, 19.0°, 22.3°, and 24.2°. In another alternative, as part of a twenty-fourth embodiment, DUR-Ca is of crystalline Form F, characterized by at least six x-ray powder diffraction peaks at 2Θ angles selected from 9.5°. 11.3°, 12.0°, 14.0°, 17.0°, 19.0°, 22.3°, and 24.2°. In another alternative, as part of a twenty-fourth embodiment, DUR-Ca is of crystalline Form F, characterized by x-ray powder diffraction peaks at 2Θ angles selected from 9.5°, 11.3°, 12.0°, 14.0°, 17.0°, 19.0°, 22.3°, and 24.2°. In another alternative, as part of a twenty-fourth embodiment, DUR-Ca is of crystalline Form F, characterized by at least three, at least four, at least five, at least six, or at least seven x-ray powder diffraction peaks at 2Θ angles recited in Table 21.

In a twenty-fifth embodiment, DUR-Ca crystalline Form F is at least 70% a single crystalline form by weight, at least 80% a single crystalline form by weight, at least 90% a single crystalline form by weight, at least 95% a single crystalline form by weight, or at least 99% a single crystalline form by weight optionally characterized by the XRPD peaks recited above in the twenty-fifth embodiment. Alternatively, as part of a twenty-fifth embodiment, DUR-Ca crystalline Form F is present in pure crystalline form optionally characterized by the XRPD peaks recited above in the twenty-fifth embodiment.

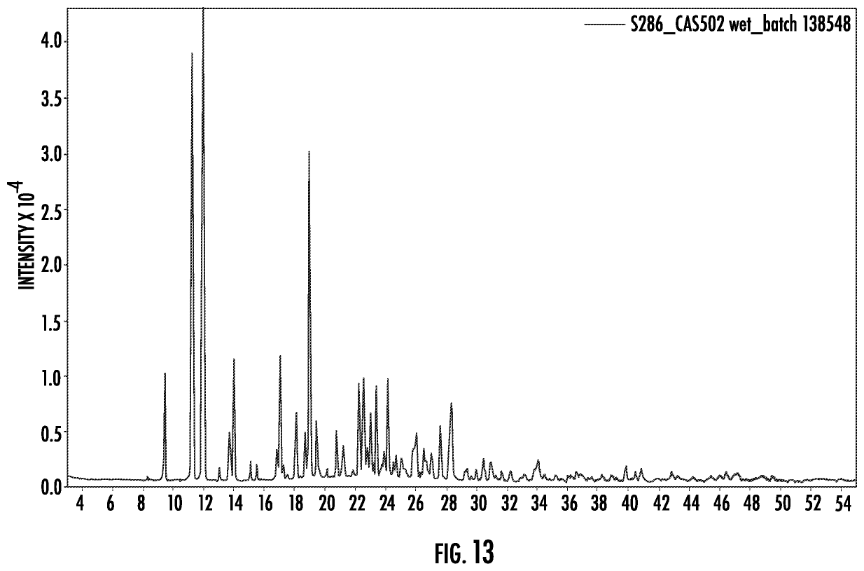

In a twenty-sixth embodiment, DUR-Ca crystalline Form F is characterized by an X-ray powder diffraction pattern substantially similar to FIG. 13.

Also provided herein are methods for preparing DUR-Ca, said methods comprising reacting DUR-TBA with calcium chloride in a solvent such as ethanol to provide DUR-Ca. In one aspect, the DUR-Ca form by the disclosed methods is crystalline Form A or B or C or F as described herein (e.g., in any one of the fifteenth to twenty-sixth embodiments).

Also provided herein are methods for preparing DUR-TEA, said methods comprising reacting a hydroxyurea compound of the structural formula

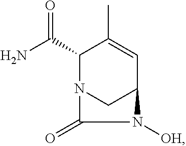

with a sulfur trioxide complex (e.g., sulfur trioxide pyridine complex, sulfur trioxide triethylamine complex, sulfur trioxide N,N-dimethylformamide complex, and the like) and triethylamine to form DUR-TEA. In one aspect, the DUR-TEA synthesized by the disclosed methods is crystalline Form A as described herein (e.g., in any one of the fourth to sixth embodiments). In one aspect, the sulfur trioxide complex used in the preparation of DUR-TEA is sulfur trioxide pyridine complex. In one aspect, the reaction of the hydroxyurea compound with the sulfur trioxide pyridine complex and trimethylamine occurs in a solvent such as acetonitrile. In one aspect of the methods described above for preparing DUR-TEA, the method further comprises precipitating the triethylammonium salt from solution with a co-solvent such as acetone.

Also provided are methods for preparing DUR-TBA, said methods comprising reacting DUR-TEA with tetrabutylammonium hydrogen sulfate and sodium dihydrogen phosphate to form DUR-TBA. In one aspect, the DUR-TBA and/or DUR TEA is of crystalline Form A as described herein (e.g., in any one of the fourth to sixth and/or nineth to twelfth embodiments). In one aspect of the methods described above for preparing DUR-TBA, the method further comprises precipitating the tetrabutylammonium salt from a solvent such as acetone.

Also provided are methods for preparing DUR-Ca, said methods comprising reacting DUR-TBA with calcium chloride to form DUR-Ca. In one aspect, the DUR-TBA and/or DUR-Ca is of crystalline Form B as described herein (e.g., in any one of the ninth to twelfth and/or fifteenth to seventeenth embodiments). In one aspect, the DUR-Ca is of crystalline Form A as described herein (e.g., in any one of the eighteenth to twentieth embodiments). In one aspect, the DUR-Ca is of crystalline Form C as described herein (e.g., in any one of the twenty-first to twenty-third embodiments). In one aspect, the DUR-Ca is of crystalline Form F as described herein (e.g., in any one of the twenty-fourth to twenty-sixth embodiments). In one aspect of the methods described above for preparing DUR-Ca, the reaction is completed in a solvent such as ethanol.

Also provided are methods for preparing DUR-Na, said methods comprising reacting either DUR-TEA or DUR-TBA with sodium ion exchange resin to form DUR-Na. In one aspect, the DUR-TEA and/or DUR TBA is of crystalline Form A as described herein (e.g., in any one of the third to sixth and/or nineth to twelfth embodiments).

Also provided are methods for preparing DUR-Na, said method comprising reacting DUR-Ca with sodium carbonate to form DUR-Na. In one aspect, the DUR-Ca is of crystalline Form B as described herein (e.g., in any one of the fifteenth to seventeenth embodiments). In one aspect, the DUR-Ca is of crystalline Form A as described herein (e.g., in any one of the eighteenth to twentieth embodiments). In one aspect, the DUR-Ca is of crystalline Form C as described herein (e.g., in any one of the twenty-first or twenty-third embodiments). In one aspect, the DUR-Ca is of crystalline Form F as described herein (e.g., in any one of the twenty-fourth to twenty-six embodiments).

The following examples are intended to be illustrative and are not intended to be limiting in any way to the scope of the disclosure.

### Exemplification

TABLE 1
List of Abbreviations for Solvents

| Abbreviation | Solvent | Abbreviation | Solvent |
|---|---|---|---|
| ACN | Acetonitrile | MeOH | Methanol |
| IPA | 2-Propanol | MeOAc | Methyl Acetate |
| ACN/MeCN | Acetonitrile | MTBE | Methyl tert- |
| | | | Butyl Ether |
| DCM | Dichloromethane | THF | Tetrahydrofuran |
| EtOH | Ethanol | TFE | Trifluoroethanol |
| EtOAc | Ethyl Acetate | 2-MeTHF | 2- |
| | | | Methyltetrahydrofuran |
| MEK | Methyl Ethyl | n-PA | n-Propanol |
| | Ketone | | |
| TFA | Trifluoroacetic Acid | DMSO | Dimethyl sulfoxide |
| TEA | Triethylamine | DMF | Dimethylformamide |
| PE | Petroleum ether | DMA/DMAc | Dimethylacetamide |
| IPOAc | Isopropyl acetate | | |

TABLE 2
List of Instruments and Abbreviation
Instruments

| Full Name | Abbreviation |
|---|---|
| Differential Scanning Calorimetry | DSC |
| Dynamic Vapor Sorption | DVS |
| High Performance Liquid Chromatography | HPLC |
| Nuclear Magnetic Resonance | NMR |
| X-ray Powder Diffraction | XRPD |
| Thermogravimetric Analysis | TGA |
| Liquid Chromatography Mass Spectrometry | LCMS |

TABLE 3
List of Measurement Units
Units

| Full Name | Abbreviation |
|---|---|
| Celsius | C |
| Degrees | ° |
| Equivalents | eq. |
| Gram | g |
| Hour | h |
| Kelvin | K |
| Liters | L |
| Milligrams | mg |
| Milliliters | mL |
| Minute | min |
| Milliamp | mA |
| Kilovolt | kV |
| Relative Humidity | RH |
| Room temperature | RT |
| Second | sec |
| volume | vol. |
| Volume ratio | v/v |
| Watt | W |
| Weight | wt. |
| Weight Percentage | wt. % |

### Experimental Data for Xrpd and Dsc Methods

XRPD method for DUR-TBA, DUR-Ca Form B, Form F

Analyses are performed from 2θ=3° to 50° by default. X-ray powder analysis diffraction were carried out in transmission mode unless mentioned otherwise. The samples (a few milligrams) are introduced with being slightly crushed in 1 mm diameter glass capillaries to avoid preferential orientation. The capillaries are sealed to avoid contact with air. The analysis is performed in transmission mode by using a focusing X-ray mirror with divergence slits and anti-scatter slits (aperture) 0.5°, on an Empyrean diffractometer from PANalytical Company equipped with a copper anticathode tube (wavelength λ Kα1=1.54060 Å/Kα2=1.54443 Å) and with a PIXcel 1D detector with anti-scatter slits of 7.5 mm. The calibration of the analytical instrument is checked before each analytical batch according to quality system. This table summarizes the experimental conditions of measurements.

| Measurement type | Single scan | Anti-scatter slit | |
|---|---|---|---|
| Sample offsets | | Name: | Fixed slit 1/2° |
| 2Theta (°): | 0.0100° | Type: | Fixed |
| Used wavelength | | Height (mm): | 0.76 |
| Intended wavelength | | Divergence slit | |
| type: | | | |
| Kα1 | | Name: | Fixed slit 1/2° |
| Kα1 (Å): | 1.540598 | Distance to sample | 140 |
| | | (mm): | |
| Kα2 (Å): | 1.544426 | Type: | Fixed |
| Kα2/Kα1 intensity | 0.50 | Height (mm): | 0.76 |
| ratio: | | | |
| Kα (Å): | 1.541874 | Angle (°): | 0.4354 |
| Kβ (Å): | 1.392250 | Beam knife | |
| Incident beam path | | Name: | Beam knife for linear |
| | | | detectors |
| Radius (mm): | 240.0 | Diffracted beam | |
| | | path | |
| X-ray tube | | Radius (mm): | 240.0 |
| Name: | Empyrean XRD tube Cu LFF | Anti-scatter slit | |
| S/N: | HR DK430404 | | |
| Anode material: | Cu | Name: | Anti-scatter slit 7.5 mm |
| | | | (PIXcel) |
| Voltage (kV): | 45 | Type: | Fixed |
| Current (mA): | 40 | Height (mm): | 7.50 |
| Focus type: | Line | Soller slit | |
| Length (mm): | 12.0 | Name: | Soller slits 0.02 rad. |
| width (mm): | 0.4 | Opening (rad.): | 0.02 |
| Take-off angle (°): | 2.2 | Detector | |
| X-ray mirror | | Name: | PIXcel1D detector |
| Name: | Focusing X-ray mirror for Cu | Type: | RTMS detector |
| | radiation | | |
| Crystal: | | PHD - Lower level | 25 |
| | | (%): | |
| Name: | W/Si | PHD - Upper level | 70 |
| | | (%): | |
| Type: | Graded | Mode: | Scanning |
| Shape: | Parabolic | Active length (°): | 3.3473 |
| Acceptance angle (°): | 0.800 | Sample mode | Capillary |
| Length (mm): | 55.3 | Scan | |
| Soller slit | | Scan axis: | Gonio |
| Name: | Soller slits 0.02 rad. | Scan range (°): | 3-50 |
| Opening (rad.): | 0.02 | Step size (*): | 0.0131 |
| Mask | | Scan mode: | Continuous |
| Name: | Fixed incident beam mask 10 | Counting time (s): | 120 |
| | mm | | |
| Width (mm): | 10.00 | | |

### XRPD Method for DUR-TEA, DUR-Ca Form A, Form C

Instrument: Bruker D8 Advance X-ray Powder Diffractometer
Method parameter:
   Diffractometer setting:
      Goniometer type: Theta/Theta
      Sample stage: standard rotating stage
      Tube parameters: voltage 40 kV, current 40 mA
Scan parameter
   Rotation speed: 30./min
   Scan angle: 3· ˜ 40* (2θ)
   Scan step: 0.02° (20)
   Scan speed: 0.1s/step
Sample preparation:

Take appropriate amount of tested sample into the sample pan, and then planished with spoon. Then tested with parameters above.

DSC method A (for DUR-TBA, DUR-TEA, DUR-Ca crystalline Form A and C)
Instrument: TA DSC Q200
Method parameter:
   Sensor: DSC (differential scanning calorimetry)
   Crucible: Gold, 25 μL with open lid
   Ramp 10° C./min from 20° C. to 450° C.
   Sample purge flow (N2): 50 ml/min
   Pan: Pinhole pan
   Mode: Standard
   Heating rate: 10K/min
Sample preparation:
Weigh 1˜3 mg sample into pinhole pan and shake slightly to make the sample surface flat, test use method.
   TGA method A (for DUR-TBA, DUR-TEA, DUR-Ca crystalline Form A and C)
Instrument: TA TGA Q500
Method parameter:
   Sensor: TGA (thermogravimetry)
   Crucible: Aluminum 25 μL with open lid
   Ramp 10° C./min from 30° C. to 300° C.
   Sample purge flow (N2): Balance at 40 ml/min and for the sample at 60 ml/min
   Pan: Open aluminum
   Mode: TGA 1000° C.
Sample preparation:
Place appropriate sample amount in a tared aluminum pan, weigh automatically and insert the pan into the TGA furnace follow the method.
   TGA & DSC method B (for DUR-Ca crystalline Form B and F)
Instrument: STA 449C Jupiter Netzsch
Method parameter:
   Sensor: TGA/DSC (thermogravimetry/differential scanning calorimetry)
   Crucible: Aluminum 25 μL with open lid
   Sample purge flow: Nitrogen, 50 mL/min
   Temperature: 25° C. to 400° C.
   Heating rate: 4K/min
Sample preparation:
Weigh 4-6 mg sample into open lid and shake slightly to make the sample surface flat, test use method.

### Summary of Crystalline Salts

As stated above, prior processes for generating DUR-Na included the use of a phosphonium salt which was then subjected to ion-exchange resin to form DUR-Na. The problem with this method was that phosphonium salt is not crystalline (making it difficult to work with), its purity is less than 95%, and it is not amenable to large-scale production. In an effort to solve this problem, a salt screen of DUR was carried out to identify crystalline salts with acceptable properties that could serve as a replacement for the phosphonium salt of DUR used in the prior process. See e.g., Example 10 of WO 2013/150296.

Since DUR is readily degraded by virtue of the free acid, the salt screen was carried out using salt exchange with crystalline DUR-TBA salt, which was a crystalline anhydrate and was soluble in most solvents.

Amorphous salts were initially prepared from six counter-ions (N-methyl-D-glucamine, tromethamine, NH₄⁺, Zn²⁺, Na⁺ and Ca²⁺) on a small scale using an ion exchange resin method, followed by freeze-drying to isolate XRPD amorphous solids. The ion exchange method was very time consuming with low yields and many of the salts contained residual TBA, even with multiple passes through the ion exchange column.

A focused crystallization screen of the amorphous salts did not find crystalline material, except for DUR-Ca. Attempts were undertaken to form DUR-Ca via salt metathesis by slurry reaction of DUR-TBA salt with six calcium salts (CaCl₂), CaBr₂, Ca(BF₄)₂. Ca(OAc)₂, Calcium D-gluconate and Calcium citrate) to find an alternative method to the ion exchange resin, which is very time consuming and costly to scale up. Solids isolated from most of the counter-ions were composed of starting materials and proved non crystalline. Eventually, after extensive experimentation, it was found that the salt exchange from TBA to Ca worked well in EtOH, where DUR-TBA and CaCl₂) are soluble, and the DUR-Ca crystallized from the solution. Upon further experimentation and extensive polymorph screen, two polymorph forms were identified and confirmed. Crystalline form A is initially formed and unstable in certain solvent systems, and is converted to a more stable crystalline form C.

We also found serendipitously that TEA salt of DUR (DUR-TEA) is also a good crystalline solid. However, efforts to find crystalline salts with other amines were fruitless. The pyridine salt is not stable and cannot be isolated as stable solid. A few other amine salts, conceivably useful as pharmaceutically appropriate salts, such as tromethamine, ammonia, N-methyl-D-glucamine, meglumine, lysine, choline, ornithine, proved to be not crystalline. A range of crystallization experiments were carried out, including evaporations, ambient temperature slurries, vapor stress at ambient temperature and temperature cycling, in many different solvents, or solvent mixtures, using crystalline DUR-Ca, DUR-TBA, and DUR-TEA salts as seeds. Under all the conditions, no crystalline solids were formed.

The technology described herein is further illustrated by the following examples which in no way should be construed as being further limiting. It should be understood that this invention is not limited in any manner to the particular methodology, protocols, and reagents, etc., described herein and as such can vary. The terminology used herein is for the purpose of describing particular embodiments only and is not intended to limit the scope of the present invention, which is defined solely by the claims.

### Detailed Conditions

### Ion Exchange Resin Experiments

Six salts were generated from DUR-TBA (see synthesis below) using ion exchange resin and included sodium, calcium, ammonium, zinc, tromethamine and N-methyl-D-glucamine. Experiments were carried out at a scale of 30-350 mg. Solutions of DUR-TBA in water were prepared and eluted through a column containing 74-274 mole equivalents of Amberlyst 15 (wet)-H or Dowex 50WX2 ion exchange resin, loaded with the desired counter ion. The solution was eluted slowly through the column under gravity. In some instances, a second pass through the resin was carried out to drive the exchange to completion. The column was washed with water and the combined eluent was frozen with liquid nitrogen or dry-ice and freeze-dried to obtain the salts as a solid. Experiments were first carried out on a small scale and if successful, were repeated on a larger scale.

In general, the salt conversion process was very time consuming as elution was carried out under gravity and elution rate was kept slow to improve product purity and yield. However, many of the salts contained residual TBA, even after multiple passes through the resin column and appeared to be particularly problematic for divalent counter-ions. In addition, Dowex resin appeared to facilitate degradation. Freeze drying of the sodium and N-methyl-D-glucamine salts was problematic, as the frozen solution thawed several times during freeze drying. Therefore, these had to be further diluted with water, which also extended lyophilization time.

The sodium, calcium, ammonium, and zinc salts were able to be scaled up for crystallization screens, but the tromethamine and N-methyl-D-glucamine degraded upon scale up. The lyophilized salts were composed of XRD amorphous powders.

### Salt Metathesis Experiments

Salt metathesis experiments were completed on 8 counter-ions (choline, lysine, magnesium, N-methyl-D-glucamine (meglumine), ornithine, potassium, tromethamine, and calcium). Experiments were carried out on a 20-40 mg scale. A 25 mg/mL solution of durlobactam tetrabutylammonium salt was prepared in various solvents and added to a smaller vial containing 1-2 mole equivalents of a co-former. A stirring bar was added to each vial, which was purged with nitrogen before sealing. The reactions were stirred in darkness for up to 7 days.

### Choline Salt Experiments

Salt metathesis slurries were set up on an approximately 30 mg scale. Reactions were stirred for several days but samples showed only the presence of choline chloride, as indicated in Table 4 below. The reaction mixtures were dried under a nitrogen stream, yielding gels and some specks of birefringent material. Analysis of these samples indicated a mixture of choline chloride and amorphous material. Attempts were made to dry the gels under vacuum for several days at ambient temperature but no improvement in crystallinity was observed visually. The inability to form the choline salt may be related to low solubility of the choline salt in the solvents used.

TABLE 4
Salt Metathesis Screen with Choline Chloride

| Solvent | Conditions | XRPD |
|---|---|---|
| (30:1) IPA:DMSO | RT slurry →dried N2 | Choline chloride |
| (12:1) EtOH:MeOAc | RT slurry →dried N2 | Amorphous + choline |
| | | chloride |
| (50:5:2) | RT slurry →dried N2 | Amorphous + choline |
| acetone:EtOAc:DMSO | | chloride |

### Lysine Salt Experiments

Attempts were made to form the durlobactam lysine salt via salt metathesis slurries and crash precipitation experiments with lysine hydrochloride. Reactions were set up in polar protic solvents due to the low solubility of lysine hydrochloride in most organic solvents. A crash precipitation experiment was unsuccessful in forming the lysine salt. Most experiments yielded lysine hydrochloride when analyzed by XRPD, as shown in Table 5 below.

TABLE 5
Salt Metathesis Screen with Lysine Hydrochloride

| Solvent | Conditions | XRPD |
|---|---|---|
| (60:1) Acetone:water | RT slurry | lysine HCl |
| THF | RT slurry | lysine HCl |
| Acetonitrile | RT slurry | — |
| (3:1) Water:IPA | RT slurry | — |
| Water | crash precipitation | — |

### Magnesium Salt Metathesis Experiments

Attempts were made to form the durlobactam magnesium salt via salt metathesis slurries and crash precipitation experiments with magnesium sulfate, chloride, or stearate. Most experiments yielded MgCl₂, MgSO₄, or Mg stearate when analyzed by XRPD, as shown in Table 6 below.

TABLE 6
Salt Metathesis Screen with Magnesium
Sulfate, Chloride, or Stearate

| Salt | Solvent | Conditions | XRPD |
|---|---|---|---|
| MgCl₂ | (30:1) Acetone:Formamide | RT slurry | MgCl₂ |
| MgCl₂ | (30:1) DCM:Formamide | RT slurry | MgCl₂ |
| MgCl₂ | (30:1) Ethanol:Formamide | RT slurry | Yellow Gel |
| MgCl₂ | (29:1) Acetone:Formamide | RT slurry | MgCl₂ |
| MgCl₂ | (29:1) Ethanol:Formamide | RT slurry→dried | Gel |
| | | N2→vacuum dry | |
| MgCl₂ | Water | crash ppt | Solution |
| Mg | Acetone | RT slurry | Mg stearate |
| stearate | | | |
| Mg | Ethyl Lactate | RT slurry | Mg stearate |
| stearate | | | |
| MgSO₄ | (30:1) Ethyl Lactate:Water | RT slurry | MgSO₄ |

### N-Methyl-D-Glucamine Salt Experiments

Attempts were made to form the durlobactam N-methyl-D-glucamine salt via salt metathesis slurries and crash precipitation experiments using N-methyl-D-glucamine hydrochloride. All experiments yielded NMDG HCl, as shown in Table 7 below.

TABLE 7
Salt Metathesis Screen with N-methyl-D-glucamine Hydrochloride

| Solvent | Conditions | XRPD |
|---|---|---|
| Water | crash precipitation | NMDG HCl |
| (60:1) IPA:DMSO | RT slurry | NMDG HCl |
| (1:12) Ethanol:MeOAc | RT slurry | NMDG HCl |
| (5:50:1) Acetone:EtOAc:DMSO | RT slurry | NMDG HCl |

### Ornithine Salt Experiments

Attempts were made to form the durlobactam ornithine salt via salt metathesis slurries and crash precipitation experiments using ornithine hydrochloride. Most experiments yielded ornithine HCl, as shown in Table 8 below.

TABLE 8
Salt Metathesis Screen with Ornithine Hydrochloride

| Solvent | Conditions | XRPD |
|---|---|---|
| Ethanol | RT slurry | Ornithine HCl |
| 60:1 Acetone:water | RT slurry | Ornithine HCl |
| 60:1 THF:water | RT slurry | Ornithine HCl |
| Acetonitrile | RT slurry | — |
| 3:3:1 ACN:water:IPA | RT slurry | Recycled |
| 20:1 EtOAc:MeOH | RT slurry | ornithine HCl |
| Water | crash precipitation | Solution |

### Potassium Salt Experiments

Attempts were made to form the durlobactam potassium salt via salt metathesis slurries and crash precipitation experiments using either potassium acetate or potassium chloride. Most experiments yielded gels, as shown in Table 9 below.

TABLE 9
Salt Metathesis Screen with Potassium
Acetate or Potassium Chloride

| Salt | Solvent | Conditions | XRPD |
|---|---|---|---|
| KOAc | Ethanol | RT slurry→dried | Amorphous |
| | | N2→vacuum dry | (gel + solid) |
| KOAc | Ethyl Lactate | RT slurry→dried | Brown gel |
| | | N2→vacuum dry | |
| KCl | (30:1) | RT slurry→dried | Gel |
| | Acetone:Formamide | N2→vacuum dry | |
| KCl | (60:1) | RT slurry→dried | Gel |
| | DCM:MeOH | N2→vacuum dry | |
| KCl | (120:5:4) | RT slurry→dried | Gel |
| | EtOH:Water:Formamide | N2→vacuum dry | |
| KCl | (29:1) | RT slurry→dried | Gel |
| | Acetone:Formamide | N2→vacuum dry | |
| KCl | (29:1) | RT slurry→dried | Gel |
| | EtOH:Formamide | N2→vacuum dry | |
| KCl | Water | crash precipitation | KCl |

### Tromethamine Salt Metathesis Experiments

Attempts were made to form the durlobactam tromethamine salt via salt metathesis slurries and crash precipitation experiments using tromethamine hydrochloride. Most experiments yielded tromethamine HCl, as shown in Table 10 below.

TABLE 10
Salt Metathesis Screen with Tromethamine Hydrochloride

| Solvent | Conditions | XRPD |
|---|---|---|
| (30:1) | RT slurry→dried | Gel and solid |
| IPA:DMSO | N2→vacuum dry | |
| (1:12) | RT slurry | Tromethamine |
| Ethanol:MeOAc | | HCl |
| (2:5:50) | RT slurry | Tromethamine |
| DMSO:Acetone:EtOAc | | HCl |
| Water | crash precipitation | solution |

### Calcium Salt Metathesis Experiments

Attempts were made to form the durlobactam calcium salt via salt metathesis slurries using a variety of calcium salts. Most of the experiments yielded no crystalline material or the calcium salt starting material, as shown in Table 11 below. While Ca (BF₄)₂ yielded a variety of crystalline structures, these were typically disordered and were not amenable to scale up procedures. From this screen, only CaCl₂ in EtOH or IPA provided consistent and scalable crystallization.

TABLE 11
Salt Metathesis Screen with various Calcium Salts

| Calcium Salt | Solvent | Conditions | XRPD |
|---|---|---|---|
| CaCl₂ | DCM | RT slurry | N/A |
| CaCl₂ | ACN | RT slurry | N/A |
| CaCl₂ | THF | RT slurry→dried | Type 1 + 6 |
| | | N₂ | |
| CaCl₂ | EtOH | RT slurry | Type 1 |
| CaCl₂ | EtOAc:EtOH | RT slurry | N/A |
| | (8:1) | | |
| CaCl₂ | IPA | RT slurry | Type 3 |
| CaCl₂ | MeOH | RT slurry | N/A |
| CaCl₂ | EtOH:Water | RT slurry | N/A |
| CaBr₂ | EtOH | RT slurry | N/A |
| CaBr₂ | IPA | RT slurry | Type 3 |
| CaBr₂ | DCM | RT slurry→dried | Amorphous |
| | | N₂ | |
| CaBr₂ | ACN | RT slurry | Type 4 |
| CaBr₂ | EtOAc:EtOH | RT slurry | N/A |
| | (8:1) | | |
| Ca(BF₄)₂ | ACN | RT slurry | Type 7 |
| Ca(BF₄)₂ | Acetone | RT slurry | Type 1 |
| Ca(BF₄)₂ | IPA | RT slurry→dried | Type 7 |
| | | N₂ | |
| Ca(BF₄)₂ | EtOH | RT slurry | Type 8 |
| Ca(BF₄)₂ | THF | RT slurry | Type 7 |
| Ca(OAc)₂ | EtOH:Water | RT slurry | Type 2 |
| Ca(OAc)₂ | IPA | RT slurry | Ca(OAc)₂ |
| Ca(OAc)₂ | THF | RT slurry | Ca(OAc)₂ |
| Ca(OAc)₂ | DCM | RT slurry→dried | Ca(OAc)₂ |
| | | N2 | |
| Ca(OAc)₂ | ACN | RT slurry | Ca(OAc)₂ |
| Calcium D- | EtOH:Water | RT slurry | Calcium D- |
| gluconate | | | gluconate |
| Calcium D- | ACN:Water | RT slurry | Calcium D- |
| gluconate | | | gluconate |
| Calcium D- | DCM:Water | RT slurry | Calcium D- |
| gluconate | | | gluconate |
| Calcium D- | IPA:Water | RT slurry | Calcium D- |
| gluconate | | | gluconate |
| Calcium D- | EtOAc:EtOH | RT slurry→dried | Calcium D- |
| gluconate | (8:1) | N₂ | gluconate |
| Calcium Citrate | IPA | RT slurry | Calcium Citrate |
| Calcium Citrate | THF | RT slurry | Calcium Citrate |
| Calcium Citrate | EtOH | RT slurry | Calcium Citrate |
| Calcium Citrate | EtOAc:EtOH | RT slurry→dried | Calcium Citrate |
| | (8:1) | N₂ | |

### Crystallization Screens

As the salt metathesis screen led to few successful salts and no crystalline materials, only the sodium, calcium, ammonium, and zinc salts made through ion exchange (method 1) were tested for crystallinity as described below.

### Crystallization Screen of Durlobactam Ammonium Salt

A range of crystallization experiments were carried out on durlobactam ammonium salt, including evaporations, ambient temperature slurries, slurries seeded with DUR-TBA, vapor stress at ambient temperature and temperature cycling.

The screening methods are as follows:

Slow Evaporation—A solution of durlobactam salt was prepared in each solvent. The solution was evaporated in a fume hood at ambient temperature in a vial under a flow of nitrogen. The resulting solids were analyzed by XRPD.

Slurry Experiment—Sufficient durlobactam salt was added to a given solvent until undissolved solids remained at the stated temperature. The vial was sealed, and the slurry was maintained at the selected temperature and agitated by shaking for up to 14 days. Samples were examined daily by polarized light microscopy for crystallinity.

Vapor Stressing—Aliquots of durlobactam salts were weighed into virgin glass vials. These vials were placed uncapped into larger vials containing 500 μL of a selected solvent. The larger vials were capped and stored at 20 or 40° C. The samples were examined visually by polarized light microscopy.

Temperature Cycling—The test solvent (1 mL) was added to a sample of durlobactam salt (˜3-10 mg) at ambient temperature and 5-16 cycles of the following temperature program was performed using the Clarity crystallization station:
Heat from 0° C. to 20° C. at 0.5° C./min
Hold at 20° C. for 1 min
Cool to 0° C. at 0.1° C./min
Hold at 0° C. for 1 min
No stirring

Seeding Experiments—Slurries of durlobactam salts were seeded with crystalline salt DUR-TBA or crystalline salt DUR-Ca. The material was slurred for several days at 20 or 60° C. and examined by polarized light microscopy for crystallinity.

Sonication—Sufficient durlobactam salt was added to a selected solvent until excess undissolved solids remained. The mixture was sonicated at 30% intensity using a Cole-Parmer 130 W ultrasonic processor and a pulsed program. In cases where no solids precipitated at ambient temperature, the sample was stored at 4° C. for 18 hours. All solids recovered from these experiments were analyzed using XRPD.

The results of the various experiments are detailed in Table 12 below and show that no method produced crystalline material.

TABLE 12
Crystallization screen of Durlobactam Ammonium Salt

| | Screening | |
|---|---|---|
| Solvent | method | Observations |
| Ethanol | slow evaporation | yellow oil |
| DMSO | slow evaporation | pale yellow solution |
| TFE | slow evaporation | pale yellow oil |
| acetone/ACN | slurry (60° C.) | orange solid |
| EtOAc/MTBE | slurry (60° C.) | no significant crystallinity by |
| | | microscopy after 12 days |
| THF | slurry (60° C.) | pale yellow solid |
| DCM | slurry (60° C.) | pale orange solid |
| Ethanol | vapor stress | oil |
| ACN | vapor stress | no significant crystallinity by |
| | | microscopy after 16 days |
| EtOAc | vapor stress | no significant crystallinity by |
| | | microscopy after 16 days |
| MTBE | vapor stress | no significant crystallinity by |
| | | microscopy after 16 days |
| MEK | vapor stress | XRPD: amorphous |
| IPOAc | vapor stress | XRPD: amorphous |
| methanol | vapor stress | mostly dissolved |
| anisole | vapor stress | no significant crystallinity by |
| | | microscopy after 16 days |
| EtOH-pentane | vapor stress | orange oil |
| methanol-MTBE | vapor stress | reddish solid |
| acetone | slurry (RT) | orange solid |
| THF | slurry (RT) | yellow solid |
| ethyl acetate | slurry (RT) | clear, pale yellow, glassy solid |
| MTBE:EtOH | slurry (RT) | clear, colorless, glassy solid |
| (9:1 v/v) | | |
| ACN:MeOH | slurry (RT) | pale yellow/orange haze |
| (9:1 v/v) | | |
| DCM | temp cycle | clear, pale yellow, glassy solid |
| MEK | temp cycle | off white, glassy solid |
| ACN/EtOH | temp cycle | clear, pale yellow, glassy solid |
| (9:1 v/v) | | |
| EtOAc/EtOH | temp cycle | clear, pale yellow, glassy solid |
| (9:1 v/v) | | |
| MTBE/MeOH | temp cycle | yellow, glassy solid |
| (9:1 v/v) | | |
| IPOAc | temp cycle | clear, pale yellow, glassy solid |

### Crystallization Screen of Durlobactam Calcium Salt (DUR-Ca)

Initial slurry experiments were completed using the durlobactam calcium salt. The results of the various experiments are detailed in Table 13 below, and show that only the use of Ethanol or EtOAc produced crystalline material, more comprehensive crystallization screen was later performed and the results are shown in Table 13.

TABLE 13
Initial Crystallization screen of Durlobactam Calcium Salt

| | Screening | | |
|---|---|---|---|
| Solvent | method | Observations | XRPD |
| Acetone | Slurry (RT) | yellow solid. small, | — |
| | | white needles | |
| EtOH | Slurry (RT) | clusters of small, | Crystalline-Form A |
| | | white needles | (This later changed |
| | | (B + E) | to Form C from |
| | | | another screen shown |
| | | | in Table 19) |
| THF | Slurry (RT) | yellow solid. | — |
| | | Patches B + E | |
| ACN | Slurry (RT) | yellow solid + | — |
| | | white needles | |
| EtOAc | Slurry (RT) | yellow with long | Crystalline-Form A |
| | | needles | (This later changed |
| | | | to Form C from |
| | | | another screen shown |
| | | | in Table 19) |
| DCM | Slurry (RT) | yellow, glassy | — |
| | | solid + fibers | |
| MTBE | Slurry (RT) | yellow, glassy | — |
| | | solid + fibers | |
| EtOH-water | Slurry (RT) | small white needles | — |
| EtOH-water | Slurry (RT) | fine white | Most amorphous |
| | | needles + | |
| | | amorphous | |

Note:
B = birefringence,
E = extinction by crossed-polarized light,
RT = room temperature

### Crystallization Screen of Durlobactam Zinc Salt

Slurry and vapor stressing experiments were completed using the durlobactam zinc salt. The results of the various experiments are detailed in Table 14 below and show that no method produced crystalline material.

TABLE 14
Crystallization screen of durlobactam Zinc Salt

| | Screening | |
|---|---|---|
| Solvent | method | Observations |
| ethanol | vapor stress | Dissolved. |
| EtOAc | vapor stress | No crystallinity by |
| | | microscopy |
| DCM | slurry (RT) | Yellow, glassy material + |
| | | fibers |
| EtOAc | slurry (RT) | Yellow, glassy material + |
| | | fibers |
| ACN | slurry (RT) | yellow, glassy material + |
| | | cloudy suspension |
| THF | slurry (RT) | Pale yellow solid + haze. |
| ethanol | slurry (RT) | off-white, cloudy suspension |
| THF | slurry (RT) seeded | Pale yellow haze. No seeds |
| | with 2026-099-02 | visible |
| | (crystalline AZD2514 | |
| | Ca salt) | |

Despite extensive salt screening methods, crystallization methods, and other conditions, including metal salts (calcium, zinc, magnesium, and potassium) and amines (tromethamine, ornithine, N-methyl-D-glucamine, lysine, choline, and ammonia), only the calcium salt proved to be a scalable and crystalline material. In addition to the calcium salt, the triethylamine and tetrabutylammonium salts were also found to be scalable and crystalline, as is discussed in the subsequent examples.

### Preparation and Characterization of Durlobactam Tetrabutylammonium Salt (DUR-TBA) Form A

### Method 1

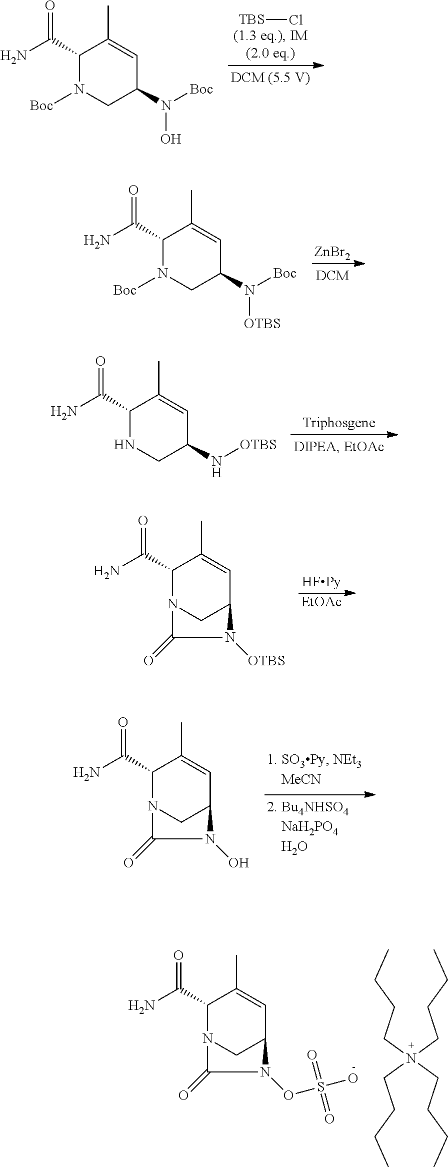

To a solution of tert-butyl (3R,6S)-3-((tert-butoxycarbonyl) (hydroxy) amino)-6-carbamoyl-5-methyl-3,6-dihydropyridine-1 (2H)-carboxylate (for synthesis of this compound, see WO2018/53215) (110 kg, 1.0 eq.) and imidazole (40.65 kg, 2.0 eq.) in DCM (634.5 kg, 4.3 V) at 0±5° C. was added a solution of TBSCl (58.5 kg, 1.3 eq) in DCM (148 kg, 1.0 V). The reaction was stirred at least 16 hours at 0° C. and washed with water three times (first washed with 555 kg of water, then second and third times washed with 333 kg of water each). After the third wash, the organic phase was distilled to remove residual water. DCM (5V) was added and distilled. This DCM addition/distillation was repeated until the water content in the organic phase is ≤0.5% by KF. HPLC indicated a purity of 99.5%. Solution was used without further purification.

To the above solution of tert-butyl (3R,6S)-3-((tert-butoxycarbonyl) ((tert-butyldimethylsilyl)oxy) amino)-6-carbamoyl-5-methyl-3,6-dihydropyridine-1 (2H)-carboxylate in DCM at 25±5° C. was added ZnBr₂ (269.1 kg, 4.0 cq.) in portions. After addition, the solution was stirred for 24 hours. Afterwards, a solution of NH₄Cl (16 cq.)/NH₄OH (16 eq.) in water (Prepared by mixing 255.5 kg of NH₄Cl with 325 kg of 25% NH₄OH in 1450 kg of water) was added. The mixture was stirred at 10±5° C. for at least 2 h, and then allowed to settle for at least 1 h.

The organic phase was transferred onto a solution of NH₄CI (10 eq)/NH₄OH (10 cq.) in water (prepared by mixing 160 kg of solid NH₄Cl with 203.5 kg of 25% NH₄OH in 1450 kg of water). The mixture was stirred at 20° C.±5° C. for at least 1 hour. The mixture was then allowed to settle for at least 30 minutes.

The organic phase was transferred onto an NH4Cl 2% w/V solution (previously prepared by mixing 58 kg of solid NH₄Cl with 2901 kg of water). The mixture was stirred at 20° C.±5° C. for at least 30 minutes and then allowed to settle for at least 30 minutes. The organic phase was washed 5 times with water at 20° C.±5° C. 8V of DCM were then distilled at atmospheric pressure. 4V of ethyl acetate were loaded, and solvent was distilled off. This process was repeated one more time.

At the end of the distillation, 4V of ethyl acetate was loaded once more to generate (2S,5R)-5-(((tert-butyldimethylsilyl)oxy) amino)-3-methyl-1,2,5,6-tetrahydropyridine-2-carboxamide in ethyl acetate solution. HPLC indicated a purity of 96.3%. Solution was used without further purification

To the solution of (2S,5R)-5-(((tert-butyldimethylsilyl)oxy) methyl)-3-methyl-1,2,5,6-tetrahydropyridine-2-carboxamide in EtOAc was added additional EtOAc (complement up to 30V), water (83 kg, 1 V), and DIEA (150 kg, 4.0 eq.). This solution was cooled to 0° C. and a solution of triphosgene (30 kg, 0.33 eq.) in EtOAc (261 kg. 3.5 V) was added over 4 hours. The solution was warmed to RT and stirred for 5 hours. Afterwards, the reaction mixture was washed twice with water (10V) and then washed with a saturated solution of NaCl (5V). Organic phase was concentrated to distill 27 V of ethyl acetate. 10 V of n-heptane was reloaded, then 8-9 V are distilled under vacuum. After distillation, the mixture was cooled to 20±5 oC and then the solid is filtered, washed twice with 1 V of mixture of ethyl acetate/heptane (1/10). The crude product was slurred in water (4 V), filtered, and washed with water (1 V), dried at 30±5 oC to give (2S,5R)-6-((tert-butyldimethylsilyl)oxy)-3-methyl-7-oxo-1,6-diazabicyclo[3.2.1]oct-3-ene-2-carboxamide.HPLC indicated a purity of 99.9%.

To a solution of (2S,5R)-6-((tert-butyldimethylsilyl)oxy)-3-methyl-7-oxo-1,6-diazabicyclo[3.2.1]oct-3-ene-2-carboxamide (32.2 kg, 1.0 eq.) in EtOAc (130.7 kg, 4.5 V) at 5±5° C. was added a solution of HF·Py (19.2 kg, 16.4% HF, 1.5 eq.) in EtOAc The addition equipment was rinsed with EtOAc (0.87 kg). After addition, the reaction was allowed to warm to 25±5° C. and stirred for 4 hours. The precipitate was collected and washed with EtOAc (29.58 kg, 1.0 V). The filter cake was added to EtOAc (59.16 kg, 2.0 V) and stirred for at least 2 hours, filtered, washed with ethyl acetate (29.58 kg. 1.0 V) HPLC indicated a purity of 100%. The solid was dried at 20±5° C. and used in next step without further purification.

To a solution of (2S,5R)-6-hydroxy-3-methyl-7-oxo-1,6-diazabicyclo[3.2.1]oct-3-ene-2-carboxamide (36 kg. 1 eq) in acetonitrile (74 kg. 94.1 L, 2.6 V) at 15±2° C. was added SO₃Py (46.5 kg. 1.6 eq.) portion wise followed by TEA (29.5 kg, 1.6 cq). After addition, the line for addition of TEA was rinsed with acetonitrile (0.4 V) and charged to the reaction mixture. The reaction mixture was stirred until starting material was consumed (in about 5 hours).

The reaction mixture was cooled to 3+3° C. and was slowly added to a pre-prepared cold solution (3° C.) of Bu₄NHSO₄ (62.0 kg. 1.05 cq) and NaH₂PO₄-H₂O (26.5 kg, 1.05 eq) in water (360 kg. 10 V). The resulting mixture was stirred at 3±3° C. for at least 4 hours and was warmed to 20±5° C., and was extracted with DCM (238.5 kg, 180 L. 5 V).

The organic phase was isolated. Aqucous phase was extracted with DCM (238.5 kg. 5V). The combined organic phases were washed with a solution of NaH₂PO₄•H₂O (7.6 kg, 0.3 cq) in water (180 kg, 5V), and concentrated to approx. 5 V. Acetone (853 kg. 1080 L. 30 V) was added in portions. The resulting mixture was concentrated to approximately 5 V. The solvent exchange with acetone (853 kg. 1080 L. 30V) was repeated one more time.

EtOAc (368.0 kg. 408 L, 4.6V, the first portion, pre-cooled to −5±5° C.) and crystalline tetrabutylammonium salt of Durlobactam seeds (360 g, 1% weight) were added. The reaction mass was stirred at 10±3° C. for 1 hour, cooled to −5±3° C. over 3-4 hours, stirred for additional minimum of 2 hours, and additional EtOAc (368.0 kg, 408 L, 4.6 V, the second portion, pre-cooled to −5±5° C.) was added. The suspension was stirred at −5±5° C. for 6 hours. Solid was collected by filtration, washed with EtOAc (2×130 kg (4 V)) and washed with n-Heptane (2×93 kg. 3.8 V). The solid was dried on the filter with nitrogen blow for at least 48-72 hours. HPLC indicated a purity of 100.0%.

¹H-NMR δ (400 MHZ, DMSO-D₆), δ 7.79 (1H, s, 1 H of NH₂), 7.32 (1H, s, 1 H of NH₂), 6.05 (1H, brs, CH), 4.09 (1H, s, CH), 4.02 (1H, s, CH), 3.68 (1H, m, 1 H of ring CH₂), 3.20 (8H, m, 4×CH₂), 3.07 (1H, m, 1 H of ring CH₂), 1.61 (3H, s, CH₃), 1.56 (8H, m, 4×CH₂), 1.35 (8H, m, 4×CH₂), 0.95 (12H, m, 4×CH₃) ppm.

### Method 2

To a solution of tetrabutylammonium chloride (93.0 g, 1.0 eq) in water (1.0 L, 10 V) at 0° C. was added DUR-Na (100.0 g, 1.0 eq, 334.0 mmol). The reaction was stirred 2 hours at RT. Afterwards, DCM (500.0 mL, 5.0 V) was added to the reaction and stirred an additional 30 minutes. The layers were separated, and the organic layer collected. The aqueous layer was extracted 1× with DCM (500.0 mL, 5.0 V).

The aqueous layer was cooled to 0° C. and additional tetrabutylammonium chloride (18.7 g, 0.2 eq) was added. The reaction was stirred for 1-2 hours. Afterwards, DCM (500.0 mL, 5.0 V) was added to the reaction and stirred for additional 30 minutes. The layers were separated, and the organic layer collected. The aqueous layer was extracted 1× with DCM (500.0 mL, 5.0 V).

The combined organic layers were concentrated to approx. 6.5 V. Acetone (3.0 L, 30.0 V) was added, and the solution concentrated to approx. 6.5V. EtOAc (2.0 L, 20.0 V) was added, and the solution was cooled to 0° C. followed by the addition of more EtOAc (4.0 L, 40.0 V). The solution was stirred for 18 hours at 0° C. The precipitated solid was collected and washed with EtOAc (200.0 mL, 2.0 V) then dried at no more than 35° C. for 24 hours.

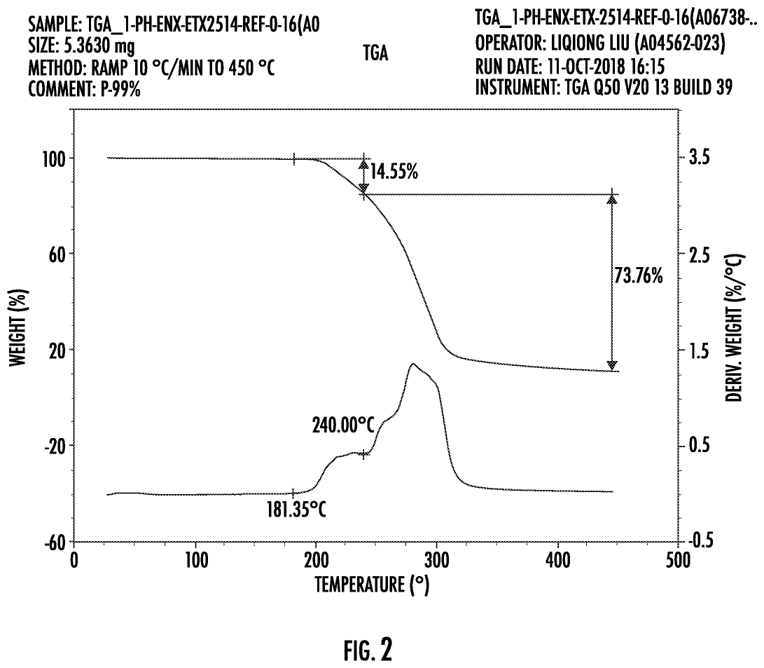

DUR-TBA crystalline Form A was characterized by XRPD (FIG. 1 and Table 15) and TGA and DSC (FIG. 2). Peaks with relative intensities of less than 1% are not reported.

TABLE 15
Peak list for XRPD pattern of DUR-TBA Form A

| Angle | Relative | d value |
|---|---|---|
| (2θ°) | intensity (%) | (Å) |
| 7.323 | 100 | 12.06134 |
| 8.468 | 16.3 | 10.43319 |
| 8.696 | 13.3 | 10.15969 |
| 10.257 | 16 | 8.61681 |
| 10.587 | 1.4 | 8.34900 |
| 11.222 | 2.2 | 7.87804 |
| 12.159 | 5 | 7.27281 |
| 12.727 | 6 | 6.94970 |
| 13.571 | 3.9 | 6.51937 |
| 13.787 | 1.4 | 6.41772 |
| 14.671 | 2.5 | 6.03309 |
| 15.321 | 1.8 | 5.77841 |
| 15.450 | 3.6 | 5.73046 |
| 15.860 | 1.8 | 5.58330 |
| 16.030 | 1.9 | 5.52454 |
| 16.449 | 5.1 | 5.38454 |
| 17.649 | 3.1 | 5.02117 |
| 17.985 | 3 | 4.92795 |
| 18.569 | 4.8 | 4.77440 |
| 18.658 | 3.7 | 4.75172 |
| 19.168 | 1.8 | 4.62640 |
| 19.538 | 6.7 | 4.53960 |
| 19.630 | 4.7 | 4.51872 |
| 20.083 | 5 | 4.41771 |
| 20.418 | 1.4 | 4.34601 |
| 20.821 | 5.5 | 4.26288 |
| 21.399 | 6.5 | 4.14889 |
| 21.679 | 4.8 | 4.09594 |
| 21.911 | 3.6 | 4.05306 |
| 22.177 | 3.6 | 4.00511 |
| 22.568 | 4.6 | 3.93664 |
| 22.937 | 1.5 | 3.87405 |
| 23.362 | 1.9 | 3.80457 |
| 23.931 | 5.5 | 3.71545 |
| 24.374 | 1.5 | 3.64892 |
| 25.499 | 1.8 | 3.49034 |
| 25.916 | 1.3 | 3.43508 |
| 26.107 | 1.1 | 3.41041 |

### Preparation and Characterization of Durlobactam Triethylamine Salt (DUR-TEA) Form A

To a solution of (2S,5R)-6-hydroxy-3-methyl-7-oxo-1,6-diazabicyclo[3.2.1]oct-3-ene-2-carboxamide (243 g, 1 eq) in acetonitrile (730 mL, 3V) at 10° C. was added SO₃Py (255 g, 1.3 eq.) portion wise followed by TEA (163 g, 1.3 eq). After addition, the reaction was stirred for least 18 hours, until starting material was consumed. Acetone (3.7 L, 15V) was added. The reaction mixture was cooled to −40° C. and the resulting mixture was stirred for at least 18 hours. Solid was collected by filtration, washed with acetone/ACN (480 mL, 2V, 5/1 ratio) and dried under vacuum at 25-30° C. for at least 24 hours. HPLC purity: 99.2%

¹H-NMR δ (400 MHZ, DMSO-D₆), 1.20 (9H, m), 2.50 (3H, s), 3.12 (7H, m), 3.67 (1H, d), 4.01 (1H, m), 4.09 (1H, s), 6.05 (1H, m), 7.32 (1H, s), 7.79 (1H, s); ¹³C-NMR (400 MHZ, in DMSO-D₆) 9.12, 20.50, 46.28, 56.79, 66.07, 126.01, 135.48, 168.67, 170.43 ppm. IR (cm⁻¹): 3442.19, 3333.79, 3070.15, 1775.26, 1691.35, 1328.65, 1274.34, 1240.85, 1159.491057.57, 1016.24, 753.72593.113

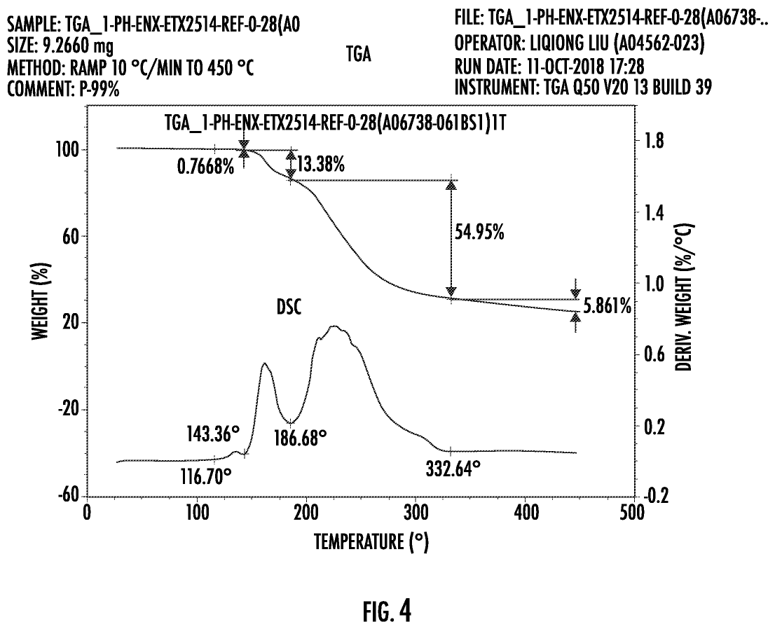

DUR-TEA crystalline Form A was characterized by XRPD (FIG. 3 and Table 16) and TGA and DSC (FIG. 4). Peaks with relative intensities of less than 1% are not reported.

TABLE 16
Peak list for XRPD pattern of DUR-TEA Form A

| Angle | Relative | d value |
|---|---|---|
| (2θ°) | intensity (%) | (Å) |
| 3.197 | 12.3 | 27.6142 |
| 9.536 | 70.1 | 9.26721 |
| 10.721 | 64.3 | 8.24511 |
| 12.675 | 48.6 | 6.97834 |
| 13.458 | 58.3 | 6.57414 |
| 15.100 | 5.6 | 5.86279 |
| 15.884 | 6.7 | 5.57512 |
| 16.701 | 10.7 | 5.30409 |
| 17.256 | 100 | 5.1346 |
| 18.034 | 36.3 | 4.91499 |
| 19.256 | 34.2 | 4.60558 |
| 20.875 | 25.7 | 4.252 |
| 21.485 | 39.5 | 4.13262 |
| 21.961 | 12.7 | 4.04408 |
| 22.559 | 63.3 | 3.93822 |
| 23.042 | 13.7 | 3.8568 |
| 24.420 | 45.2 | 3.64219 |
| 25.473 | 22.3 | 3.49391 |
| 25.873 | 18 | 3.44079 |
| 26.083 | 9.7 | 3.41359 |
| 26.324 | 4.9 | 3.38291 |
| 26.975 | 16.3 | 3.30272 |
| 27.208 | 26.4 | 3.27495 |
| 27.918 | 9.1 | 3.1932 |
| 28.539 | 8.8 | 3.12511 |
| 29.194 | 4 | 3.05653 |
| 29.432 | 7.9 | 3.03233 |
| 29.721 | 14.4 | 3.00355 |
| 30.060 | 6 | 2.97037 |
| 30.465 | 5.4 | 2.93184 |

### Preparations and Characterization of Durlobactam Calcium Salt (DUR-Ca) Form B

Into an inerted reactor, the following are loaded: CaCl₂) anhydrous (7.5 kg, 0.5 eq) and ethanol (442 kg, 8V). The reaction mixture is stirred at 20° C.±5° C. until complete solubilization and then maintained at this temperature until its use in the synthesis. Into a second inerted reactor, load the following successively: DUR-TBA (70 kg, 1 eq.) and ethanol (276.5 kg, 5 V). The reaction mixture is brought to 20° C.±5° C. and stirred at this temperature until solubilization. The calcium chloride solution (previously prepared) is then slowly added over a minimum of 1 hour (through the loading vessel with a dip tube). At the end of the addition, the reactor used for the calcium chloride solution preparation is rinsed with ethanol (41.5 kg, 0.75 V) then transferred into the synthesis reactor. The reaction mixture is maintained for a minimum of 16 hours at 20° C.±5° C. At the end of the contact, the mixture is cooled down to 0° C.±5° C. and is maintained at this temperature for a minimum of 2 hours. The mixture is filtered and washed with ethanol (110.5 kg, 2 V) that has been cooled at 0° C.±5° C. The wet cake is crystalline B, containing up to 20% EtOH as solvate. Wet DUR-Ca is slurred a first time in ethanol (276.5 kg, 5V) at 20° C.±5° C. for at least 2 hours and filtered, washed successively with ethanol (110.5 kg, 2 V) then with acetone (110 kg, 2 V), dried (35° C. under vacuum with nitrogen bleed) until constant weight prior to be analysed. Up to this stage, the solid remains as crystalline B, containing EtOH and acetone as solvate.

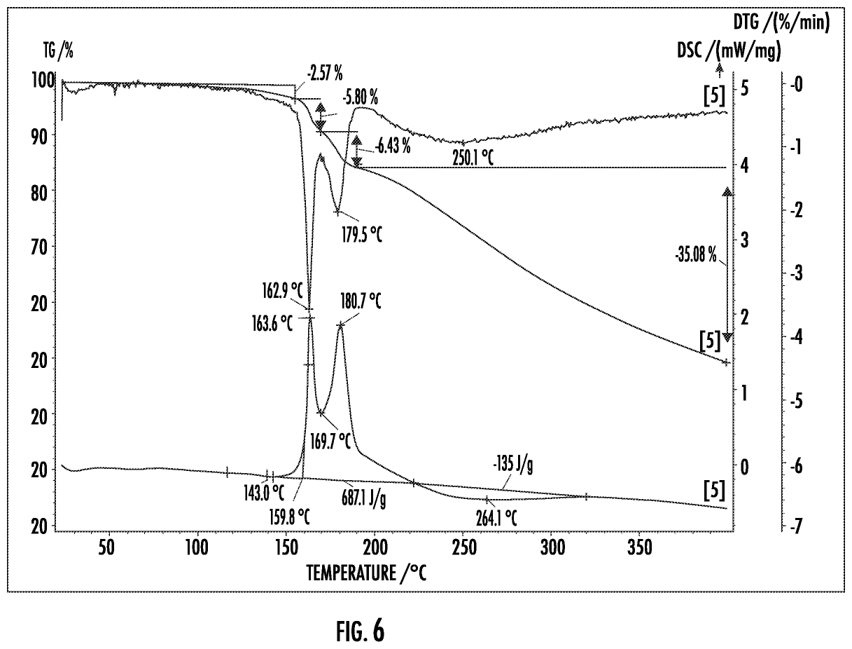

DUR-Ca crystalline Form B was characterized by XRPD (FIG. 5, Table 17) and TGA/DSC (FIG. 6). Peaks with relative intensities of less than 1% are not reported.

TABLE 17
Peak list for XRPD pattern of DUR-Ca Form B

| Angle | Relative | d value |
|---|---|---|
| (2θ°) | intensity (%) | (Å) |
| 7.164 | 20 | 12.339678 |
| 7.358 | 55 | 12.014105 |
| 9.593 | 99 | 9.220031 |
| 10.927 | 18 | 8.097217 |
| 11.991 | 29 | 7.380998 |
| 12.310 | 51 | 7.190373 |
| 12.546 | 94 | 7.055642 |
| 12.651 | 57 | 6.997317 |
| 12.658 | 62 | 6.993133 |
| 12.851 | 51 | 6.888543 |
| 14.083 | 100 | 6.288996 |
| 14.356 | 30 | 6.169793 |
| 14.747 | 7 | 6.007037 |
| 15.116 | 37 | 5.861259 |
| 15.990 | 44 | 5.542907 |
| 16.067 | 1 | 5.516379 |
| 16.331 | 53 | 5.427729 |
| 16.488 | 98 | 5.376496 |
| 16.569 | 71 | 5.350587 |
| 17.692 | 14 | 5.013175 |
| 18.013 | 5 | 4.924693 |
| 18.878 | 1 | 4.700945 |
| 19.049 | 50 | 4.659081 |
| 19.254 | 28 | 4.609983 |
| 19.476 | 28 | 4.557887 |
| 19.540 | 27 | 4.543102 |
| 19.943 | 53 | 4.452286 |
| 20.656 | 14 | 4.300121 |
| 20.988 | 40 | 4.232863 |
| 21.179 | 48 | 4.195139 |
| 21.220 | 20 | 4.187106 |
| 21.543 | 22 | 4.124938 |
| 21.570 | 16 | 4.119874 |
| 21.606 | 19 | 4.113204 |
| 21.875 | 49 | 4.063080 |
| 21.889 | 36 | 4.060623 |
| 21.955 | 9 | 4.048602 |
| 22.072 | 61 | 4.027278 |
| 22.198 | 16 | 4.004703 |
| 22.250 | 29 | 3.995444 |
| 22.487 | 72 | 3.953868 |
| 22.819 | 11 | 3.897106 |
| 23.054 | 35 | 3.857911 |
| 23.482 | 5 | 3.788619 |
| 23.748 | 37 | 3.746828 |
| 23.983 | 14 | 3.710644 |
| 24.115 | 48 | 3.690509 |
| 24.295 | 26 | 3.663618 |
| 24.403 | 26 | 3.647737 |
| 24.521 | 15 | 3.630406 |
| 24.568 | 64 | 3.623509 |
| 25.045 | 1 | 3.555610 |
| 25.245 | 19 | 3.527824 |
| 25.303 | 4 | 3.519979 |
| 25.459 | 32 | 3.498668 |
| 25.475 | 40 | 3.496574 |
| 25.868 | 8 | 3.444285 |
| 25.939 | 29 | 3.434992 |
| 26.230 | 49 | 3.397579 |
| 26.260 | 26 | 3.393830 |
| 26.595 | 48 | 3.351810 |
| 26.700 | 48 | 3.338805 |
| 26.879 | 13 | 3.317024 |
| 27.139 | 25 | 3.285870 |
| 27.189 | 42 | 3.279917 |
| 27.524 | 33 | 3.240764 |
| 27.841 | 6 | 3.204513 |
| 27.873 | 17 | 3.200929 |
| 27.904 | 32 | 3.197399 |
| 27.988 | 15 | 3.188049 |
| 28.124 | 1 | 3.172908 |
| 28.538 | 28 | 3.127804 |
| 28.682 | 6 | 3.112480 |
| 28.822 | 26 | 3.097722 |
| 28.866 | 18 | 3.093037 |
| 28.944 | 5 | 3.084900 |
| 29.055 | 20 | 3.073325 |
| 29.140 | 22 | 3.064616 |
| 29.746 | 8 | 3.003527 |
| 30.065 | 34 | 2.972353 |

### Preparations and Characterization of Durlobactam Calcium Salt (DUR-Ca) Form A

Method A: To a solution of CaCl₂) (282.5 g. 0.5 eq) in anhydrous EtOH (26.4 L. 10 V) was added dropwise a solution of DUR-TBA (2.64 kg. purity of 88.6% by Q-NMR. 1.0 eq.) in EtOH (13.2 L. 5 V) at ambient temperature. After complete addition, the reaction mixture was stirred at 15° C. for 40 hours. The reaction mixture was cooled to 0-5° C. and stirred for 4 hours. Solid was collected by centrifugation and washed with EtOH (2 V). The wet cake was slurred in EtOH (6 V) at 25-30° C. for 3 hours. Wet cake was collected by centrifugation and washed with EtOH (2 V). Wet solid was collected by centrifugation and slurred with EtOAc (12 V) at 25-30° C. for about 132 hours. Solid was collected by centrifugation and dried in oven until residual solvent in H-NMR≤2.5% to give DUR-Ca. 2.04 kg, 98% purity by HPLC % Area, 56% yield, crystalline Form A.

Method B: Into an inerted reactor, the following are loaded: CaCl₂) anhydrous (7.5 kg, 0.5 eq) and ethanol (442 kg, 8V). The reaction mixture is stirred at 20° C.±5° C. until complete solubilization and then maintained at this temperature until its use in the synthesis. Into a second inerted reactor, load the following successively: DUR-TBA (70 kg, 1 eq.) and ethanol (276.5 kg. 5 V). The reaction mixture is brought to 20° C.±5° C. and stirred at this temperature until solubilization. The calcium chloride solution (previously prepared) is then slowly added over a minimum of 1 hour (through the loading vessel with a dip tube). At the end of the addition, the reactor used for the calcium chloride solution preparation is rinsed with ethanol (41.5 kg. 0.75 V) then transferred into the synthesis reactor. The reaction mixture is maintained for a minimum of 16 hours at 20° C.±5° C. At the end of the contact, the mixture is cooled down to 0° C.±5° C. and is maintained at this temperature for a minimum of 2 hours. The mixture is filtered and washed with ethanol (110.5 kg, 2 V) that has been cooled at 0° C.±5° C. Wet DUR-Ca is slurred a first time in ethanol (276.5 kg, 5V) at 20° C.±5° C. for at least 2 hours and filtered. The cake is washed successively with ethanol (110.5 kg, 2 V) then with acetone (110 kg, 2 V). Wet DUR-Ca and acetone (384. Kg, 7 V) are loaded in the reactor then 1 equivalent (2.43 kg) of water (PUW) is added in 10 minutes minimum at 20° C.±5° C. The reaction mixture is heated to reflux (56° C.±5° C.) and stirred for 30 minutes at this temperature. The mixture is then cooled down to 20° C.±5° C. in 1 h, stirred for 1 hour, filtered and washed with acetone (110 kg, 2 V). DUR-Ca is dried under vacuum at <35° C. max until constant mass is met to give crystalline Form A, which typically contains ˜1%-5% acetone.

¹H-NMR (400 MHZ, DMSO-D₆), δ 1.61 (3H, s), 3.06 (1H, m), 3.66 (1H, d), 4.02 (1H, m), 4.09 (1H, s), 6.05 (1H, m), 7.33 (1H, s), 7.80 (1H, s) ppm.

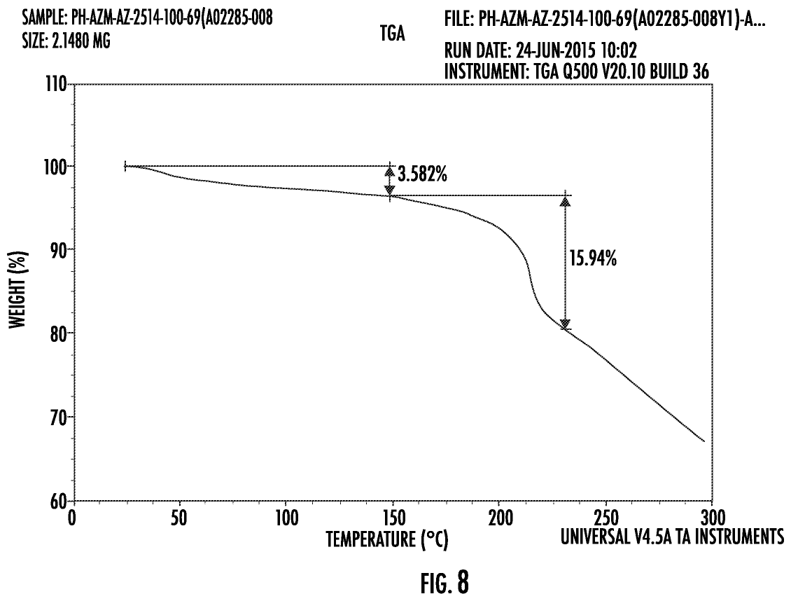

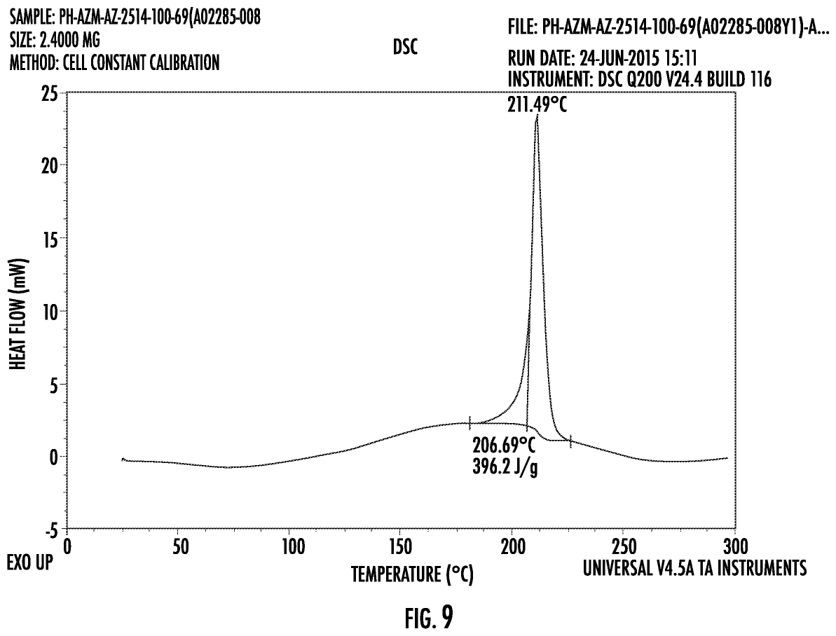

DUR-Ca crystalline Form A was characterized by XRPD (FIG. 7 and Table 18) and TGA (FIG. 8) and DSC (FIG. 9). Peaks with relative intensities of less than 1% are not reported.

TABLE 18
Peak list for XRPD pattern of DUR-Ca Form A

| Angle | Relative | d value |
|---|---|---|
| (2θ°) | intensity (%) | (Å) |
| 4.482 | 15.9 | 19.70102 |
| 7.770 | 91.7 | 11.36844 |
| 8.966 | 100 | 9.85496 |
| 11.854 | 43 | 7.46002 |
| 13.428 | 37.5 | 6.58876 |
| 15.530 | 12.2 | 5.70131 |
| 16.157 | 80.9 | 5.48128 |
| 17.849 | 4.4 | 4.9654 |
| 19.549 | 53.2 | 4.53721 |
| 20.532 | 21.2 | 4.32232 |
| 22.467 | 8.4 | 3.95408 |
| 23.762 | 13.4 | 3.74155 |
| 25.026 | 39.5 | 3.55534 |
| 25.817 | 3.6 | 3.44817 |
| 27.382 | 17.9 | 3.25449 |
| 28.127 | 17.8 | 3.16997 |
| 28.481 | 12.6 | 3.13142 |
| 31.619 | 5.8 | 2.8274 |

### Preparation and Characterization of Durlobactam Calcium Salt (DUR-Ca) Form C

### Method A

To a solution of CaCl₂) (0.6 eq) in anhydrous EtOH (5 V) was added a solution of DUR-TBA (100 g, 1.0 eq.) in EtOH (10 V), ensuring the temperature of the reaction stays at 20±3° C. during addition. After complete addition, the reaction mixture was stirred at 20±3° C. for 16 hours. Subsequently, the reaction is cooled to 0±5° C. and stirred for at least 2 hours. The solid was collected by centrifuge and washed with EtOH (1.5 V). The filter cake was added to EtOH (4 V) and stirred for at least 4 hours at 25±5° C. The solid was collected by centrifuge and washed with EtOH (1.5 V) then IPA (1.5 V). The filter cake was added to a solution of IPOAc (4 V) and water (0.7 eq.) and stirred for at least 4 hours at 25±5° C. The solid was collected by centrifuge and washed with IPOAc (1.5 V) and dried under vacuum at 32±3° C. for at least 24 hours to give Durlobactam Calcium Salt Crystalline Form C, which typically contains 6-7% water, and less than 1% EtOH and less than 1% acetone.

### Method B:

DUR-Ca Form A was slurred in 26 solvents for 3 days. A new distinct form (assigned as Form C) was obtained in most of solvents (Table 19).

TABLE 19
Salt slurry experiments with DUR-Ca Form A

| Sample | Solvent | Result |
|---|---|---|
| Slurry 1 | Methanol | Form C |
| Slurry 2 | Ethanol | Form C |
| Slurry 3 | IPA | Form C |
| Slurry 4 | THF | Amorphous |
| Slurry 5 | EtOAc | Form C |
| Slurry 6 | Acetone | Form C |
| Slurry 7 | Isobutanol | Form C |
| Slurry 8 | Isopropyl Acetate | Form C |
| Slurry 9 | Acetonitrile | Form C |
| Slurry 10 | 2-Butanone | Form C |
| Slurry 11 | Toluene | Mixture of Form A and C |
| Slurry 12 | water | Dissociated or |
| | | decomposition |
| | | product |
| Slurry 13 | Tert-butyl methyl ether | Form C |
| Slurry 14 | Propanol | Form C |
| Slurry 15 | Isopentanol | Form C |
| Slurry 16 | Butyl acetate | Form C |
| Slurry 17 | Ethyl Formate | Form C |
| Slurry 18 | 1,4-dioxane | Amorphous |
| Slurry 19 | Butanol | Form C |
| Slurry 20 | Heptane | Form C |
| Slurry 21 | Pentane | Form C |
| Slurry 22 | Cyclohexane | Form C |
| Slurry 23 | Methylisobutyl Ketone | Form C |
| Slurry 24 | Xylene | Form C |
| Slurry 25 | Isobutyl acetate | Form C |
| Slurry 26 | Ethyl ether | Form C |

DUR-Ca Form A was slurred in acetone (SV) and water (3.5 eq) at 20±5° C. for 4-24 hours. Wet solid was collected by filtration and dried under vacuum to give DUR-Ca Form C.

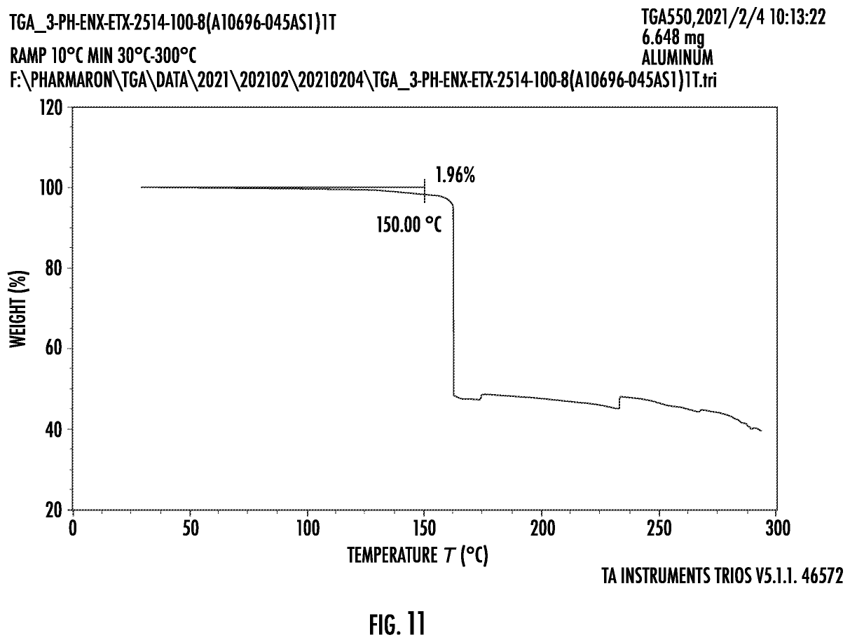

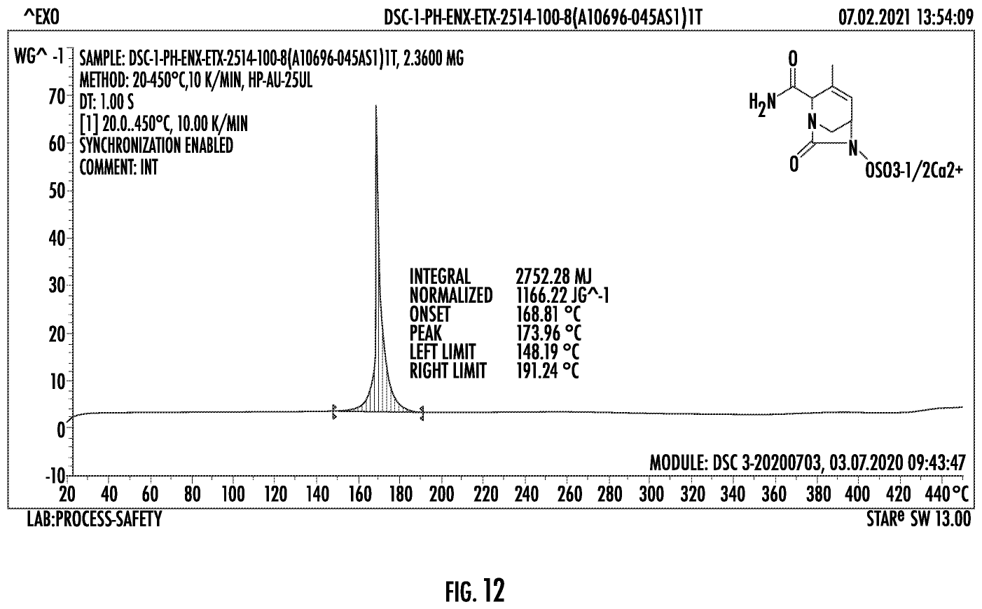

DUR-Ca crystalline Form C was characterized by XRPD (FIG. 10 and Table 20) and TGA (FIG. 11) and DSC (FIG. 12). Peaks with relative intensities of less than 1% are not reported.

TABLE 20
Peak list for XRPD pattern of DUR-Ca Form C

| Angle | Relative | d value |
|---|---|---|
| (2θ°) | intensity (%) | (Å) |
| 6.982 | 52 | 12.6496 |
| 10.393 | 2.8 | 8.5045 |
| 12.189 | 43.3 | 7.25555 |
| 14.099 | 3.9 | 6.2767 |
| 14.438 | 2.4 | 6.12976 |
| 16.081 | 54.3 | 5.50698 |
| 16.943 | 100 | 5.2289 |
| 18.745 | 20.2 | 4.73005 |
| 19.725 | 27.3 | 4.49721 |
| 20.289 | 35.6 | 4.37342 |
| 20.968 | 3.3 | 4.23335 |
| 21.292 | 4.7 | 4.16962 |
| 22.678 | 7.1 | 3.91792 |
| 23.209 | 19.8 | 3.82934 |
| 24.326 | 9.8 | 3.65607 |
| 24.643 | 3.5 | 3.60964 |
| 25.678 | 12.5 | 3.46655 |
| 25.900 | 6 | 3.43732 |
| 26.357 | 3 | 3.37872 |
| 26.865 | 40.7 | 3.31598 |
| 27.314 | 2.9 | 3.26246 |
| 28.560 | 2.6 | 3.12289 |
| 29.234 | 10.2 | 3.05239 |
| 29.632 | 3.6 | 3.01229 |
| 30.079 | 12.9 | 2.96856 |

### Preparation and Characterization of Durlobactam Calcium Salt Form F

Into an inerted reactor, the following are loaded: CaCl₂) anhydrous (7.5 kg, 0.5 eq) and ethanol (442 kg, 8 V). The reaction mixture is stirred at 20° C.±5° C. until complete solubilization and then maintained at this temperature until its use in the synthesis

Into a second inerted reactor, load the following successively: DUR-TBA (70 kg, 1 eq.) and ethanol (276.5 kg, 5V). The reaction mixture is brought to 20° C.±5° C. and stirred at this temperature until solubilization. The calcium chloride solution (previously prepared) is then slowly added over a minimum of 1 hour (through the loading vessel with a dip tube). At the end of the addition, the reactor used for the calcium chloride solution preparation is rinsed with ethanol (41.5 kg, 0.75V) then transferred into the synthesis reactor. The reaction mixture is maintained for a minimum of 16 hours at 20° C.±5° C. At the end of the contact, the mixture is cooled down to 0° C.±5° C. and is maintained at this temperature for a minimum of 2 hours. The mixture is filtered and washed with ethanol (110.5 kg, 2 V) and acetone (110 kg, 2 V) that has been pre-cooled to 0° C.±5° C. Wet DUR-Ca before slurry will be dried on the filter with a pressure of 1 bar for a minimum of 4 hours. Wet DUR-Ca and 7V (384 kg) of acetone are loaded in the reactor then 2 equivalents of water (4.86 kg) are added over a minimum of 10 minutes at 20° C.±5° C. The mixture is then stirred for 2 hours at 20° C. ±5° C., filtered and washed with acetone (110 kg, 2 V). DUR-Ca is dried on the filter with a pressure of 3 bar for a minimum of 12 hours to give crystalline Form F, which typically contains up to 20% acetone.

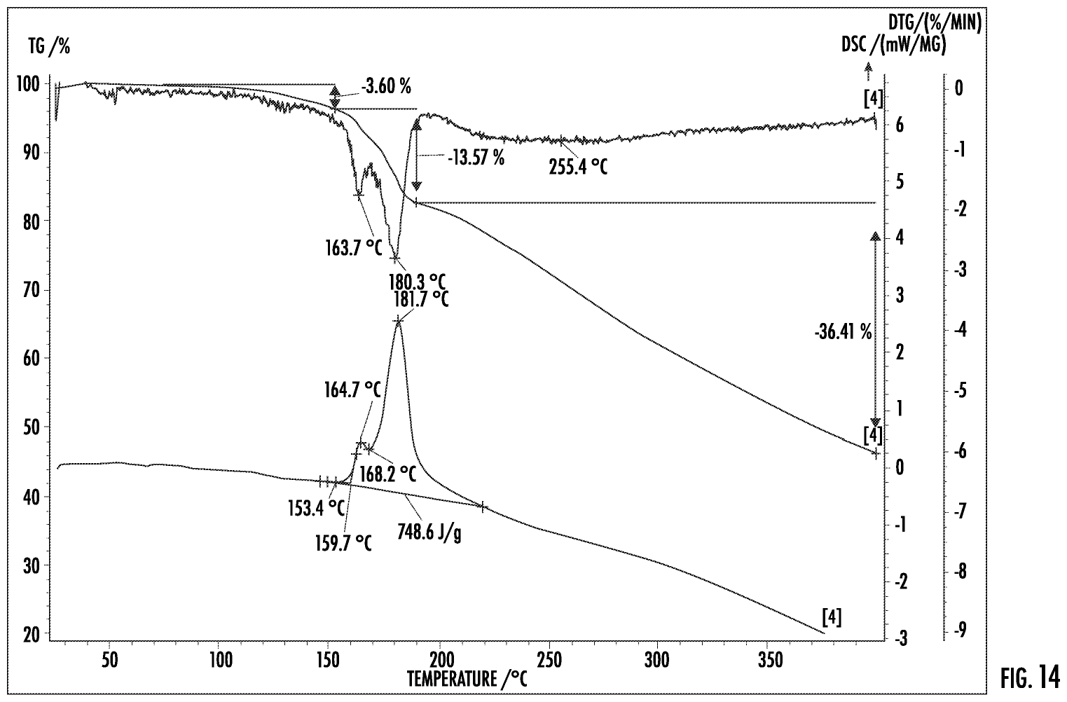

DUR-Ca crystalline Form F was characterized by XRPD (FIG. 13 and Table 21) and TGA and DSC (FIG. 14). Peaks with relative intensities of less than 1% are not reported.

TABLE 21
Peak list for XRPD pattern of DUR-Ca Form F

| Angle | Relative | d value |
|---|---|---|
| (2θ°) | intensity (%) | (Å) |
| 8.305 | 10 | 10.646218 |
| 8.501 | 7 | 10.401567 |
| 9.463 | 47 | 9.346007 |
| 11.280 | 95 | 7.844318 |
| 11.895 | 34 | 7.440040 |
| 12.024 | 100 | 7.360692 |
| 13.086 | 17 | 6.765573 |
| 13.734 | 33 | 6.447924 |
| 14.024 | 52 | 6.315187 |
| 15.112 | 19 | 5.862686 |
| 15.125 | 9 | 5.857945 |
| 15.519 | 19 | 5.710132 |
| 15.690 | 3 | 5.648102 |
| 16.655 | 7 | 5.323088 |
| 16.846 | 27 | 5.262945 |
| 17.049 | 54 | 5.200826 |
| 17.278 | 18 | 5.132291 |
| 17.554 | 10 | 5.052273 |
| 18.115 | 40 | 4.897112 |
| 18.726 | 33 | 4.738634 |
| 18.992 | 63 | 4.673008 |
| 19.020 | 67 | 4.666193 |
| 19.475 | 38 | 4.558026 |
| 19.650 | 13 | 4.517872 |
| 20.160 | 14 | 4.404676 |
| 20.785 | 35 | 4.273724 |
| 21.179 | 17 | 4.195080 |
| 21.239 | 24 | 4.183462 |
| 21.865 | 11 | 4.065026 |
| 22.265 | 50 | 3.992910 |
| 22.562 | 35 | 3.940981 |
| 22.570 | 37 | 3.939550 |
| 22.672 | 11 | 3.922159 |
| 22.808 | 26 | 3.899028 |
| 23.013 | 20 | 3.864675 |
| 23.058 | 38 | 3.857250 |
| 23.395 | 17 | 3.802542 |
| 23.426 | 47 | 3.797596 |
| 23.775 | 17 | 3.742541 |
| 23.921 | 25 | 3.720013 |
| 24.183 | 52 | 3.680331 |
| 24.505 | 19 | 3.632725 |
| 24.703 | 24 | 3.603983 |
| 25.094 | 23 | 3.548723 |
| 25.248 | 14 | 3.527426 |
| 25.384 | 13 | 3.508903 |
| 25.435 | 3 | 3.501928 |
| 25.783 | 22 | 3.455499 |
| 25.867 | 23 | 3.444494 |
| 25.956 | 23 | 3.432859 |
| 26.039 | 31 | 3.422130 |
| 26.257 | 3 | 3.394122 |
| 26.347 | 11 | 3.382795 |
| 26.516 | 14 | 3.361579 |
| 26.540 | 26 | 3.358644 |
| 26.683 | 21 | 3.340894 |
| 26.800 | 7 | 3.326574 |
| 26.851 | 12 | 3.320456 |
| 27.047 | 21 | 3.296778 |
| 27.063 | 15 | 3.294865 |
| 27.589 | 26 | 3.233254 |
| 27.611 | 27 | 3.230716 |
| 27.670 | 20 | 3.223961 |
| 27.832 | 6 | 3.205574 |
| 28.264 | 41 | 3.157586 |
| 28.375 | 37 | 3.145486 |
| 28.401 | 19 | 3.142655 |
| 28.421 | 11 | 3.140456 |
| 28.542 | 7 | 3.127364 |
| 28.655 | 6 | 3.115341 |
| 29.246 | 17 | 3.053688 |
| 29.400 | 13 | 3.038052 |
| 29.428 | 13 | 3.035215 |
| 29.681 | 11 | 3.009957 |
| 29.983 | 18 | 2.980324 |

Different crystalline forms of DUR-Ca contain different level of solvents. The residual solvent contents are summarized in Table 22.

TABLE 22
Typical Residual Solvents in DUR-Ca Crystalline Forms

| Dur-Ca | Typical | Typical | Typical |
|---|---|---|---|
| Crystalline | EtOH % | acetone % | water % |
| Form | content | content | content |
| Form A | | 1%-5% | |
| Form B | 12%-20% | Up to 20% of EtOH and | |
| | | acetone when washed/ | |
| | | slurred with EtOH/ | |
| | | acetone before acetone/ | |
| | | water is used for slurry | |
| Form C | <1% | <1% | 5%-7% |
| Form F | | Up to 20% | |

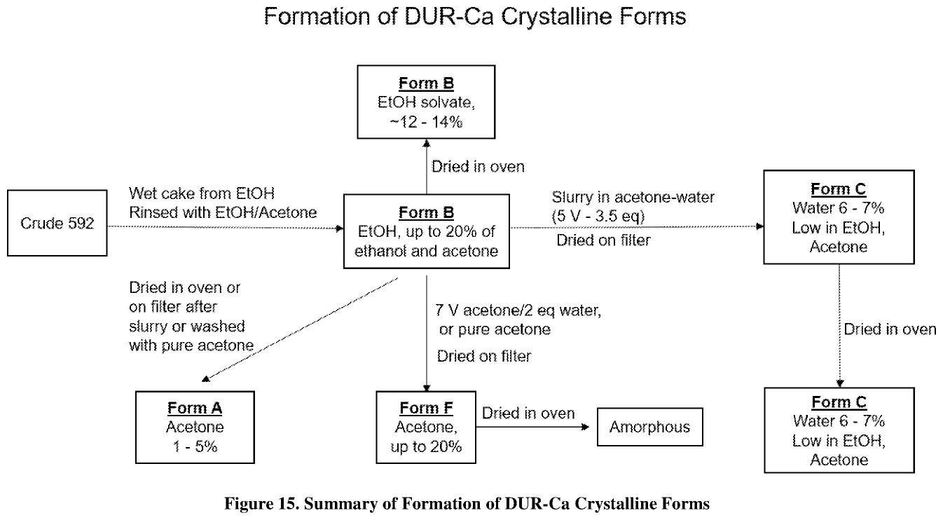

The formation of crystalline forms A, B, C, and F is summarized in FIG. 15.

Synthesis of Durlobactam Sodium Salt (DUR-Na) from Other Durlobactam Salts
Method A—Synthesis of DUR-Na from DUR-TEA

Purolite®C100E, 1375.0 g, 2500% wt was added to a NaOH solution (2.0 M, 1.0 L) and stirred at 17° C. for 12 hours. The resin was collected and washed with water until the pH was 7-9 then acidified with glacial acetic acid until the pH was 5-6.

To a solution of DUR-TEA (54.98 g, 145.28 mmol, 1.0 eq.) in water (550 mL, 10.0 V) was added the resin prepared above (275 g, 500% wt). The solution was stirred for 1 hour at 17° C. The resin was filtered off and the filtrate collected as durlobactam sodium salt.

Method B—Synthesis of DUR-Na from DUR-TBA

Amberlyst 15 (wet)-H resin (30.21 g, 57.10 mmol) was slurred in water (100 mL) and poured into a 2 cm diameter glass column (resin bed height: 21.0 cm). The resin was washed with water (150 mL). Sodium chloride (33.65 g, 575.9 mmol) was dissolved in water (540 mL) and the resulting solution was eluted slowly through the resin. The pH was monitored using pH indicator strips and was shown to change from pH5→pH1→pH5. The resin was washed with water (300 mL), and the water was allowed to run through until ˜0.5 cm remained above the resin bed.

A solution of DUR-TBA (352.5 mg, 0.6792 mmol) in water (18 mL) was prepared, and was carefully applied to the column. The solution was eluted slowly through the column under gravity. The vial containing the DUR-Na solution was rinsed with water (18 mL) and the rinse was also applied to the column. The resin was washed with a further 35 mL of water. All eluents were collected in a virgin glass jar. The combined eluent was reapplied to the column and eluted slowly under gravity. The resin was washed with water (35 mL) and the eluent collected in a virgin glass jar. The combined eluent was frozen with liquid nitrogen and freeze dried.

The product was isolated as a fluffy, white powder with static cling (189.9 mg, 93.2% recovery).

Method C-Synthesis of DUR-Na from DUR-Ca

DUR-Ca (29.0 kg, 1 equiv.) was added to a pre-cooled (0-5° C.) solution of water (87 kg, 3V) and stirred until dissolved. Afterwards, a sodium carbonate solution (4.84 kg anhydrous Na₂CO₃ in 43.6 kg of water) was slowly added (in 1 hour minimum) while the temperature was maintained below 5° C. The pH of the reaction mixture was monitored during the addition of the base to ensure that the pH didn't exceed 8.5 throughout addition. After the addition was complete, the reaction mixture was stirred at 0-5° C. for 1 hour minimum and then filtered to remove calcium carbonate that precipitated out at the end of the salt exchange. The spent calcium carbonate was rinsed with pre-cooled DI water three times (14.5 kg, 0.5 V for each wash) at 0-5° C. The combined filtrate was freeze dried to give DUR-Ca as an amorphous solid.

### Large Scale Manufacture

A comparison of the purity achieved using the disclosed processes to form DUR-Na vs. the process described in WO 2013/150296 is shown below in Table 23.

TABLE 23
HPLC purity of DUR-Na lots from different
synthesis method/process.

| | API purity (% |
|---|---|
| Synthetic Method | area in HPLC) |
| DUR-TBA to DUR-Ca to DUR-Na | 99.1% |
| API was made from durlobactam | 94.8% |
| phosphonium salt intermediate | |
| using ion exchange resin as | |
| described in WO 2013/150296. | |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A salt of a compound having the structural Formula I: 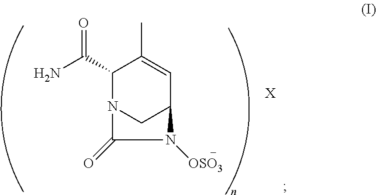
wherein
n is 1 or 2; and
X is a positively charged amine or a Ca, Mg, Zn, K, Na, Li, Cs, Ba, Rb, Sr, Fe, Co, Ni, Cu, Zn, Ag, or Au cation.

2. The salt of the compound of claim 1, wherein X is a positively charged amine or a Ca cation.

3. The salt of the compound of claim 1 or 2, wherein X is a positively charged amine.

4. The salt of the compound of any one of claims 1 to 3, wherein X is a protonated tertiary amine or a quaternary ammonium.

5. The salt of the compound of any one of claims 1 to 4, wherein X is trimethylammonium, triethylammonium, tributylammonium, triisopropylammonium, or N,N-diisopropylethylammonium.

6. The salt of the compound of any one of claims 1 to 4, wherein X is triethylammonium.

7. The salt of the compound of any one of claims 1 to 6, wherein the salt is of the structural formula:

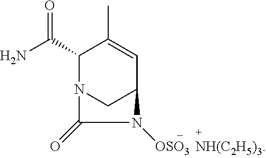

8. The salt of the compound of any one of claims 1 to 7, wherein the salt is crystalline.

9. The salt of the compound of claim 7 or 8, wherein the salt is crystalline Form A.

10. The salt of the compound of claim 9, wherein the crystalline Form A is characterized by at least three x-ray powder diffraction peaks at 2Θ angles selected from 9.5°, 10.7°, 12.7°, 13.5°, 17.3°, 22.6°, and 24.4°.

11. The salt of the compound of claim 9 or 10, wherein the crystalline Form A is characterized by at least four x-ray powder diffraction peaks at 2Θ angles selected from 9.5°, 10.7°, 12.7°, 13.5°, 17.3°, 22.6°, and 24.4°.

12. The salt of the compound of any one of claims 9 to 11, wherein the crystalline Form A is characterized by at least five x-ray powder diffraction peaks at 2Θ angles selected from 9.5°, 10.7°, 12.7°, 13.5°, 17.3°, 22.6°, and 24.4°.

13. The salt of the compound of any one of claims 9 to 12, wherein the crystalline Form A is characterized by at least six x-ray powder diffraction peaks at 2Θ angles selected from 9.5°, 10.7°, 12.7°, 13.5°, 17.3°, 22.6°, and 24.4°.

14. The salt of the compound of any one of claims 9 to 13, wherein the crystalline Form A is characterized by x-ray powder diffraction peaks at 2Θ angles 9.5°, 10.7°, 12.7°, 13.5°, 17.3°, 22.6°, and 24.4°.

15. The crystalline Form A of any one of claims 9 to 14, wherein the crystalline Form A is at least 70% a single crystalline form by weight, at least 80% a single crystalline form by weight, at least 90% a single crystalline form by weight, at least 95% a single crystalline form by weight, or at least 99% a single crystalline form by weight.

16. The salt of the compound of claim 9, wherein the crystalline Form A is characterized by an X-ray powder diffraction pattern substantially similar to FIG. 3.

17. The salt of the compound of any one of claims 1 to 4, wherein X is tetrabutylammonium, tetraethylammonium, tetramethylammonium, or tetrapropylammonium.

18. The salt of the compound of any one of claims 1 to 4 and 17, wherein X is tetrabutylammonium.

19. The salt of the compound of any one of claims 1 to 4, 17, and 18, wherein the salt is of the structural formula:

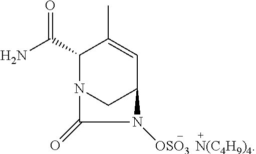

20. The salt of the compound of claim 18 or 19, wherein the salt is crystalline.

21. The salt of the compound of any one of claims 18 to 20, wherein the salt is crystalline Form A.

22. The salt of the compound of claim 21, wherein the crystalline Form A is characterized by at least three x-ray powder diffraction peaks at 2Θ angles selected from 7.3°, 8.5°, 8.7°, 10.3°, 12.7°, 19.5° and 21.4°.

23. The salt of the compound of claim 21 or 22, wherein the crystalline Form A is characterized by at least four x-ray powder diffraction peaks at 2Θ angles selected from 7.3°, 8.5°, 8.7°,10.3°, 12.7°, 19.5° and 21.4°.

24. The salt of the compound of any one of claims 21 to 23, wherein the crystalline Form A is characterized by at least five x-ray powder diffraction peaks at 2Θ angles selected from 7.3°, 8.5°, 8.7°, 10.3°, 12.7°, 19.5° and 21.4°.

25. The salt of the compound of any one of claims 21 to 24, wherein the crystalline Form A is characterized by at least six x-ray powder diffraction peaks at 2Θ angles selected from 7.3°, 8.5°, 8.7°, 10.3°, 12.7°, 19.5° and 21.4°.

26. The salt of the compound of any one of claims 21 to 25, wherein the crystalline Form A is characterized by x-ray powder diffraction peaks at 2Θ angles 7.3°, 8.5°, 8.7°,10.3°, 12.7°, 19.5° and 21.4°.

27. The salt of the compound of any one of claims 21 to 26, wherein the crystalline Form A is at least 70% a single crystalline form by weight, at least 80% a single crystalline form by weight, at least 90% a single crystalline form by weight, at least 95% a single crystalline form by weight, or at least 99% a single crystalline form by weight.

28. The salt of the compound of claim 21, wherein the crystalline Form A is characterized by an X-ray powder diffraction pattern substantially similar to FIG. 1.

29. The salt of the compound of claim 1 or 2, wherein the cation is Ca.

30. The salt of the compound of any one of claims 1, 2, and 29, wherein the salt is of the structural formula:

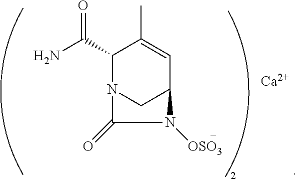

31. The salt of the compound of claim 29 or 30, wherein the salt is crystalline.

32. The salt of the compound of any one of claims 29 to 31, wherein the salt is crystalline Form A, B, C or F.

33. The salt of the compound of claim 32, wherein the crystalline Form B is characterized by at least three x-ray powder diffraction peaks at 2Θ angles selected from 9.6°, 12.5°, 12.7°, 14.1°, 16.5°, 16.6, 22.5°, and 24.6°.

34. The salt of the compound of claim 32 or 33, wherein the crystalline Form B is characterized by at least four x-ray powder diffraction peaks at 2Θ angles selected from 9.6°, 12.5°, 12.7°, 14.1°, 16.5°, 16.6, 22.5°, and 24.6°.

35. The salt of the compound of any one of claims 32, 33 and 34, wherein the crystalline Form B is characterized by at least five x-ray powder diffraction peaks at 2Θ angles selected from 9.6°, 12.5°, 12.7°, 14.1°, 16.5°, 16.6, 22.5°, and 24.6°.

36. The salt of the compound of any one of claims 32 and 33 to 35, wherein the crystalline Form B is characterized by at least six x-ray powder diffraction peaks at 2Θ angles selected from 9.6°, 12.5°, 12.7°, 14.1°, 16.5°, 16.6, 22.5°, and 24.6°.

37. The salt of the compound of any one of claims 32 and 33 to 36, wherein the crystalline Form B is characterized by at least seven x-ray powder diffraction peaks at 2Θ angles selected from 9.6°, 12.5°, 12.7°, 14.1°, 16.5°, 16.6, 22.5°, and 24.6°.

38. The salt of the compound of any one of claims 32 and 33 to 37, wherein the crystalline Form B is characterized by x-ray powder diffraction peaks at 2Θ angles 9.6°, 12.5°, 12.7°, 14.1°, 16.5°, 16.6, 22.5°, and 24.6°.

39. The salt of the compound of any one of claims 33 to 38, wherein the crystalline Form B is at least 70% a single crystalline form by weight, at least 80% a single crystalline form by weight, at least 90% a single crystalline form by weight, at least 95% a single crystalline form by weight, or at least 99% a single crystalline form by weight.

40. The salt of the compound of claim 32, wherein the crystalline Form B is characterized by an X-ray powder diffraction pattern substantially similar to FIG. 5.

41. The salt of the compound of claim 32, wherein the crystalline Form A is characterized by at least three x-ray powder diffraction peaks at 2Θ angles selected from 7.8°, 9.0°, 11.9°, 13.4°, 16.2°, 19.5°, 20.5°, and 25.0°.

42. The salt of the compound of claim 32 or 41, wherein the crystalline Form A is characterized by at least four x-ray powder diffraction peaks at 2Θ angles selected from 7.8°, 9.0°, 11.9°, 13.4°, 16.2°, 19.5°, 20.5°, and 25.0°.

43. The salt of the compound of any one of claims 32, 41 and 42, wherein the crystalline Form A is characterized by at least five x-ray powder diffraction peaks at 2Θ angles selected from 7.8°, 9.0°, 11.9°, 13.4°, 16.2°, 19.5°, 20.5°, and 25.0°.

44. The salt of the compound of any one of claims 32 and 41 to 43, wherein the crystalline Form A is characterized by at least six x-ray powder diffraction peaks at 2Θ angles selected from 7.8°, 9.0°, 11.9°, 13.4°, 16.2°, 19.5°, 20.5°, and 25.0°.

45. The salt of the compound of any one of claims 32 and 41 to 44, wherein the crystalline Form A is characterized by at least seven x-ray powder diffraction peaks at 2Θ angles selected from 7.8°, 9.0°, 11.9°, 13.4°, 16.2°, 19.5°, 20.5°, and 25.0°.

46. The salt of the compound of any one of claims 32 and 41 to 45, wherein the crystalline Form A is characterized by x-ray powder diffraction peaks at 2Θ angles 7.8°, 9.0°, 11.9°, 13.4°, 16.2°, 19.5°, 20.5°, and 25.0°.

47. The salt of the compound of any one of claims 41 to 46, wherein the crystalline Form A is at least 70% a single crystalline form by weight, at least 80% a single crystalline form by weight, at least 90% a single crystalline form by weight, at least 95% a single crystalline form by weight, or at least 99% a single crystalline form by weight.

48. The salt of the compound of claim 32, wherein the crystalline Form A is characterized by an X-ray powder diffraction pattern substantially similar to FIG. 7.

49. The salt of the compound of claim 32, wherein the crystalline Form C is characterized by at least three x-ray powder diffraction peaks at 2Θ angles selected from 7.0°, 12.2°, 16.1°, 16.9°, 19.7°, 20.3°, and 26.9°.

50. The salt of the compound of claim 32 or 49, wherein the crystalline Form C is characterized by at least four x-ray powder diffraction peaks at 2Θ angles selected from 7.0°, 12.2°, 16.1°, 16.9°, 19.7°, 20.3°, and 26.9°.

51. The salt of the compound of any one of claims 32, 49 and 50, wherein the crystalline Form C is characterized by at least five x-ray powder diffraction peaks at 2Θ angles selected from 7.0°, 12.2°, 16.1°, 16.9°, 19.7°, 20.3°, and 26.9°.

52. The salt of the compound of any one of claims 32 and 49 to 51, wherein the crystalline Form C is characterized by at least six x-ray powder diffraction peaks at 2Θ angles selected from 7.0°, 12.2°, 16.1°, 16.9°, 19.7°, 20.3°, and 26.9°.

53. The salt of the compound of any one of claims 32 and 49 to 52, wherein the crystalline Form C is characterized by at least seven x-ray powder diffraction peaks at 2Θ angles selected from 7.0°, 12.2°, 16.1°, 16.9°, 19.7°, 20.3°, and 26.9°.

54. The salt of the compound of any one of claims 32 and 49 to 53, wherein the crystalline Form C is characterized by x-ray powder diffraction peaks at 2Θ angles 7.0°, 12.2°, 16.1°, 16.9°, 19.7°, 20.3°, and 26.9°.

55. The salt of the compound of any one of claims 32 and 49 to 54, wherein the crystalline Form C is at least 70% a single crystalline form, at least 80% a single crystalline form, at least 90% a single crystalline form, at least 95% a single crystalline form, or at least 99% a single crystalline form by weight.

56. The salt of the compound of claim 32, wherein the crystalline Form C is characterized by an X-ray powder diffraction pattern substantially similar to FIG. 10.

57. The salt of the compound of claim 32, wherein the crystalline Form F is characterized by at least three x-ray powder diffraction peaks at 2Θ angles selected from 9.5°, 11.3°, 12.0°, 14.0°, 17.0°, 19.0°, 22.3°, and 24.2°.

58. The salt of the compound of claims 32 and 57, wherein the crystalline Form F is characterized by at least four x-ray powder diffraction peaks at 2Θ angles selected from 9.5°, 11.3°, 12.0°, 14.0°, 17.0°, 19.0°, 22.3°, and 24.2°.

59. The salt of the compound of any one of claims 32, 57, and 58, wherein the crystalline Form F is characterized by at least five x-ray powder diffraction peaks at 2Θ angles selected from 9.5°, 11.3°, 12.0°, 14.0°, 17.0°, 19.0°, 22.3°, and 24.2°.

60. The salt of the compound of any one of claims 32 and 57 to 59, wherein the crystalline Form F is characterized by at least six x-ray powder diffraction peaks at 2Θ angles selected from 9.5°, 11.3°, 12.0°, 14.0°, 17.0°, 19.0°, 22.3°, and 24.2°.

61. The salt of the compound of any one of claims 32 and 57 to 60, wherein the crystalline Form F is characterized by at least seven x-ray powder diffraction peaks at 2Θ angles selected from 9.5°, 11.3°, 12.0°, 14.0°, 17.0°, 19.0°, 22.3°, and 24.2°.

62. The salt of the compound of any one of claims 32 and 57 to 61, wherein the crystalline Form F is characterized by x-ray powder diffraction peaks at 2Θ angles 9.5°, 11.3°, 12.0°, 14.0°, 17.0°, 19.0°, 22.3°, and 24.2°.

63. The salt of the compound of any one of claims 32 and 57 to 62, wherein the crystalline Form F is at least 70% a single crystalline form, at least 80% a single crystalline form, at least 90% a single crystalline form, at least 95% a single crystalline form, or at least 99% a single crystalline form by weight.

64. The salt of the compound of claim 32, wherein the crystalline Form F is characterized by an X-ray powder diffraction pattern substantially similar to FIG. 13.

65. A method for preparing a calcium salt of a compound having the formula: 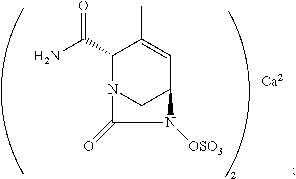
said method comprising:
reacting a tetrabutylammonium salt having the formula 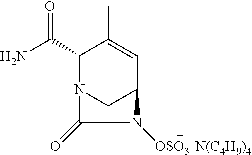
with calcium chloride to form the calcium salt.

66. The method of claim 65, wherein the tetrabutylammonium salt is reacted with calcium chloride in ethanol.

67. The method of claims 65 and 66, wherein the calcium salt is crystalline Form A, B, C or F.

68. A method for preparing a triethylammonium salt of a compound having the formula: 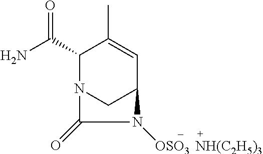
said method comprising:
reacting a hydroxyurea compound of the structural formula 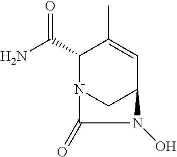
with a sulfur trioxide pyridine complex and triethylamine to form the triethylammonium salt.

69. The method of claim 68, wherein the hydroxyurea compound is reacted with sulfur trioxide pyridine complex and trimethylamine in acetonitrile.

70. The method of claim 68 or 69, further comprising precipitating the triethylammonium salt from solution.

71. The method of claim 70, wherein the triethylammonium salt is precipitated from acetone.

72. The method of any one of claims 68 to 71, wherein the triethylammonium salt is crystalline Form A.

73. A method for preparing a tetrabutylammonium salt of a compound having the formula: 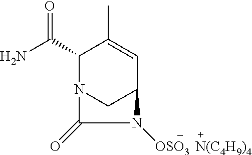
said method comprising:
reacting a triethylammonium salt of a compound having the formula: 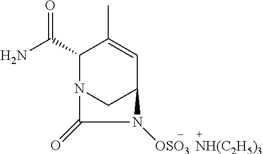
with tetrabutylammonium hydrogen sulfate and sodium dihydrogen phosphate to form the tetrabutylammonium salt.

74. The method of claim 73, further comprising precipitating the tetrabutylammonium salt from acetone.

75. The method of claim 73 or 74, wherein the tetrabutylammonium salt is crystalline Form A.

76. A method for preparing a sodium salt of a compound having the formula: 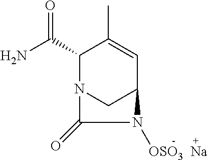
said method comprising:
reacting
i) a triethylammonium salt of a compound having the formula: 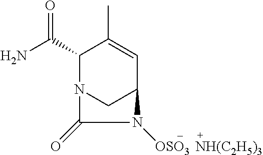
or
ii) a tetrabutylammonium salt of a compound having the formula: 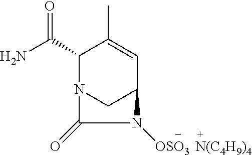
with an ion exchange resin comprising sodium to form the sodium salt.

77. A method for preparing a sodium salt of a compound having the formula: 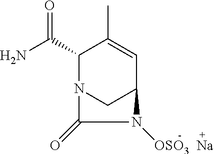
said method comprising:
reacting a calcium salt of a compound having the formula: 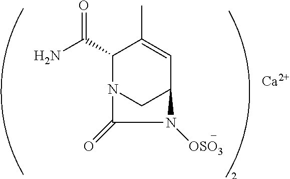
with sodium carbonate to form the sodium salt.
